SEQUENCE LISTING (2 sequences)

SEQ ID NO: 1 - protein, 36 aa, Homo sapiens
YPIKPEAPGEDASPEELNRYYASLRHYLNLVTRQRY

SEQ ID NO: 2 - protein, 34 aa, Homo sapiens
IKPEAPGEDASPEELNRYYASLRHYLNLVTRQRY

(19) United States
(12) Patent Application Publication
(10) Pub. No.: US 2008/0125360 A1
(43) Pub. Date: May 29, 2008

(54) Composition Comprising Pyy for the Treatment of Gastrointestinal Disorders

(76) Inventor: Henrik Nilsson, Kobenhavn K (DK)

Correspondence Address: BROWDY AND NEIMARK, P.L.L.C.;624 NINTH STREET, NW, SUITE 300, WASHINGTON, DC, 20001-5303, US

(21) Appl. No.: 11/596,973

(22) PCT Filed: May 18, 2005
(86) PCT No.: PCT/DK05/00327

§ 371 Date: Sep. 17, 2007

Related U.S. Application Data
(63) Continuation-in-part of application No. PCT/DK05/00111, filed on Feb. 18, 2005.

(30) Foreign Application Priority Data
May 18, 2004 (DK) PA 2004 00791
Apr. 21, 2005 (DK) PA 2005 00582

(51) Int. Cl. A61K 38/22; A61K 31/137; A61K 31/55; A61P 1/00; A61P 1/14

(52) U.S. Cl. 514/12; 514/654; 514/214.02

(57) ABSTRACT

The present invention relates to PYY or functional equivalents thereof for use in pharmaceutical compositions. The pharmaceutical compositions are in particular useful in the treatment of functional gastrointestinal disorders, such as irritable bowel disease and functional dyspepsia. The invention further relates to methods of treatment using said compositions. Further included is the combination of PYY or functional equivalents thereof with a secondary active ingredient such as an anti-emetic drug.

All patent and non-patent references cited in the application, or in the present application, are also hereby incorporated by reference in their entirety.

### FIELD OF INVENTION

The present invention relates to a composition comprising PYY for the treatment of gastrointestinal disorders. The composition of the invention is for the production of a pharmaceutical composition for the treatment of IBS, FD and/or abdominal pain. The invention further relates to a method of treatment comprising administration of said pharmaceutical composition, alone or in combination with a second active ingredient.

### BACKGROUND OF INVENTION

Gastrointestinal disorders are very common in the population. Some of these are very well characterised and thus suitable treatment regimes have been developed. It is more difficult to develop treatments for functional gastrointestinal disorder. This term refers to a disorders or diseases where the primary abnormality is an altered physiological function (the way the body works) rather than an identifiable structural or biochemical cause. Thus, these types of gastrointestinal disorders have unknown aetiology, e.g. are lacking a biological explanation. Generally, this type of disorder can not be diagnosed in a traditional way; that is, as an inflammatory, infectious, or structural abnormality that can be seen by commonly used examination, x-ray, or laboratory test.

Gastrointestinal disorders with unknown aetiology include irritable bowel syndrome (IBS) also called irritable colon, functional dyspepsia (FD) or Non-ulcer dyspepsia. Therefore the diagnosis and development of suitable treatment for this subset of gastrointestinal disorders have so far not been sufficiently successful.

### Irritable Bowel Syndrome

The predominant symptoms of IBS are abdominal pain, altered bowel habit, discomfort associated with disturbed defecation and bloating. Patients have an increased mucus and nausea and feeling of constipation and distension. The criteria for irritable bowel syndrome are pain or discomfort for 12 weeks of the previous 12 months associated with two of the following; relief with defecation, looser or more frequent stools, harder or less frequent stools, according to Rome II (se below) reviewed by Talley, N J and Spiller R (2002) and Talley, N J (2003). The symptoms may be chronic and impair the quality of life for the patient.

### Rome II Symptom Criteria for IBS

At least 12 weeks or more, which need not be consecutive, in the preceding 12 months of abdominal discomfort or pain that has two out of three features:

### 1) Relieved with defecation; and/or

### 2) Onset associated with a change in frequency of stool; and/or

### 3) Onset associated with a change in form (appearance) of stool.

Other symptoms that are not essential but support the diagnosis of IBS:
   Abnormal stool frequency (greater than 3 bowel movements/day or less than 3 bowel movements/week);
   Abnormal stool form (lumpy/hard or loose/watery stool);
   Abnormal stool passage (straining, urgency, or feeling of incomplete evacuation);
   Passage of mucus;
   Bloating or feeling of abdominal distension.

It is estimated that 10-15% of the population experience IBS symptoms.

The patients are grouped in three groups based on there predominant bowel habit (diarrhoea and/or constipation):
IBS associated with abdominal pain, fecal urgency and diarrhoea
IBS associated with abdominal discomfort, bloating and constipation
IBS associated with alternating diarrhoea and constipation

### Functional Dyspepsia

The symptoms of functional dyspepsia (FD) are partially overlapping with the symptoms of IBS. Dyspepsia refers to persistent or recurrent epigastric pain or subjective upper abdominal discomfort that may be characterized by early satiety, prostprandial fullness, or bloating. Dyspepsia is not restricted to meal-related symptoms. Patients are grouped in dysmotility-like FD and ulcer-like FD based on the prevalent symptom centred in the upper abdomen (discomfort and pain respectively). Many patients experience symptoms after meal ingestion, including epigastric discomfort, fullness and pain. Further symptoms include inability to finish a normal-sized meal, bloating, belching, nausea and vomiting (Feinle-Bisset, C. et al. 2003). The underlying mechanism of functional dyspepsia is unclear. The role of delay gastric emptying is debated and currently not the favoured model.

### Central Nervous System, Visceral Hypersensitivity and Stress

The brain-gut interaction plays a prominent role in the modulation of gut function in health and disease. The central nervous system (CNS) regulates the functions of the gastrointestinal tract, such as motility, secretion and blood flow. This occurs unperceived by the individual. In case of irregularities of the gastrointestinal an individual may experience short periods of pain. In the case of patients with gastrointestinal diseases the pain may be very strong and durable. There appears to be a connection with visceral hypersensitivity in the pathophysiology of functional gastrointestinal diseases including IBS and FD (Feinle-Bisset, C. et al. 2003). Furthermore, stress, anxiety or recall of aversive memories can enhance perception of painful events. Thus stress might have an inducing effect on hyperalgesia (Drossman D A, et al. 2002).

### Psychological Factors

Psychological and sociological factors appears relevant for the onset and severity of symptoms (Drossman D A, et al. 2002), it further appears that the most effective drugs for treatment of irritable bowel syndrome so far have been antidepressants (Talley, N. J. and Spiller R).

### Treatments

Until now treatments of gastrointestinal disorders with unknown aetiology as irritable bowel syndrome and functional dyspepsia have been based on empirical findings and aims for control of symptoms. The complexity of the symptoms indicates that a single cure will not be beneficial for all patients.

Symptoms relating to diarrhoea and constipation may be treated using laxatives an antidiarrhoeals but the success is modest. Furthermore, drugs as Loperamide may minimise diarrhoea but does not modulate the abdominal pain (review by Talley, N. J. 2003), suggesting that the irregular bowel habit and the experienced abdominal pain is not directly linked.

As mentioned above antidepressants have some effect on IBS, mostly patient with diarrhoea associated IBS may benefit from a treatment of tricyclic antidepressant, such as desipramine, nortyptiline and fluphenzine, but the trials have been of variably quality. It is further expected that selective serotonin reuptake inhibitors (SSRI) maybe useful in IBS patients with constipation (review by Talley, N. J. 2003).

### Gut Hormones

Several gut hormones have been suggested to be related to symptoms of gastrointestinal disorders. VIP, CCK, and motilin relates to the motility of the upper gastrointestinal tract whereas polypeptide YY (PYY) and Neuropeptide Y (NPY) affect the absorption in the intestine. The latter (both PYY and NPY) have been suggested as treatment for diarrhoea (U.S. Pat. No. 6,588,708) by prolonging of the residence time.

### PYY

The gut hormone peptide YY (PYY), and the neuropeptide, neuropeptide Y (NPY), are structurally related to pancreatic polypeptide (PP) (FIG. 1). PYY and NPY exert their action through NPY receptors (Y1R, Y2R, Y4R and Y5R). The PP, NPY and PYY peptides consist of 36 amino acids with an amidated C-terminal. Two forms of PYY, PYY1-36 and PYY3-36, the latter being a truncated form of the former, have been found in circulation. PYY3-36 is produced by the cleavage of PYY1-36 by the enzyme dipeptidyl peptidase IV (DPP-IV). PYY1-36 binds to and activates at least three NPY receptor subtypes (Y1, Y2 and Y5) whereas PYY3-36 is more selective for the Y2 receptor (Y2R). Only the C-terminal part of the PYY3-36 peptide is required for the binding to the Y2 receptor. Throughout this document, the notation PYY covers both PYY1-36 and PYY3-36 (Berglund, M. M. et al., 2003).

PYY was initially isolated from porcine intestine (Tatemoto, K. and Mutt, C., 1980) and named Peptide YY due to the tyrosine residues present in the N- and C-terminal of the molecule.

PYY is expressed in endocrine cells lining the gastrointestinal tract and particularly in the distal portion. PYY is secreted in response to food ingestion. Within 15 minutes the plasma level of PYY rise and the level of PYY will reach a plateau after approximately 90 min. The maximum level of PYY reached is proportional to the calories ingested, suggesting that PYY may function as a sensor of food ingestion. In addition PYY is also expressed by neurones, such as in peripheral neurons, particularly enteric neurons. Furthermore, PYY is found in a restricted set of central neurons. The expression pattern of PYY in both endocrine cells and neurons suggest that PYY may be involved in regulation of multiple functions in the individual (Ekblad, E. and Sundler, F., 2002).

A suggested role of PYY may be to regulate the secretion and absorbance of fluid and electrolytes in the gastrointestinal tract and intestine and PYY have therefore been suggested as treatment of diarrhoea (U.S. Pat. No. 6,588,708) by prolonging of the residence time. Furthermore, PYY3-36 has been suggested to be involved in the system regulating feeding behaviour. It has been found that peripheral administration of PYY3-36 inhibited food intake in rodents. Moreover, direct intra-arcuate administration of PYY3-36 inhibited food intake. A linkage between the PYY effect on feeding behaviour and the NPY 2 receptor have been suggested by the demonstration that NPY receptor Y2 null mice are resistant to the anorectic effects of peripherally administered PYY3-36 (Batterham, R. L. and Bloom, S. R., 2003).

The hypothalamic arcuate nucleus, a key brain area regulating appetite, has access to nutrients and hormones within the peripheral circulation. NPY neurons within the arcuate nucleus express the Y2R. The arcuate nucleus contains two distinct sub-groups of neurons that control food intake. On group of neurons produces NPY, which acts in the brain to stimulate feeding (Stanley, B. G. et al, 1986), whereas an adjacent subgroup of neurones produces melanocortin peptides, which act in the same brain areas as NPY, but inhibit eating (Fan, W. et al, 1997). Typically, when one of these subsets is activated, the other one is inhibited.

### REFERENCES

Batterham, R. L. and Bloom, S. R. (2003). The gut hormone peptide YY regulates appetite. Ann. N.Y. Acad. Sci. June; 994:162-8.
Berglund, M. M.; Hipskind, P. A. and Gehlert, D. R. (2003). Recent developments in our understanding of the physiological role of PP-fold peptide receptor subtypes. Exp Biol Med (Maywood). March; 228(3):217-44.
Drossman, D. A.; Camilleri, M.; Mayer, E. A.; Whitehead W E (2002). AGA technical review on irritable bowel syndrome. Gastroenterology. December; 123(6):2108-31.
Ekblad, E. and Sundler, F. (2002). Distribution of pancreatic polypeptide and peptide YY. Peptides. February; 23(2):251-61.
Fan, W.; Boston, B. A.; Kesterson, R. A.; Hruby, V. J.; Cone, R. D (1997). Role of melanocortinergic neurons in feeding and the agouti obesity syndrome. Nature. January 9; 385(6612):165-168.
Feinle-Bisset, C.; Vozzo, R.; Horowitz, M., and Talley, N. J. (2003). American Journal of Gastroenterology, January; 99 (1): 170-181
Stanley, B. G.; Kyrkouli, S. E.; Lampert, S. and Leibowitz, S. F. (1986). Peptides 7, 1189-1192).
Talley, N. J. and Spiller R (2002). Irritable bowel syndrome: a little understood organic bowel disease. The Lancet, Vol 360, August 17, 555-564.
Talley, N. J. (2003). Evaluation of drug treatment in irritable bowel syndrome. J. Clin. Pharmacol, 56, 362-369.
Tatemoto, K. and Mutt, V. (1980). Isolation of two novel candidate hormones using a chemical method for finding naturally occurring polypeptides. Nature, June 5; 285(5764):417-8.

### SUMMARY OF INVENTION

The invention relates to a composition comprising PYY for the production of a medicament for the treatment of functional gastrointestinal disorders. Said medicament may be used for the treatment of irritable bowel syndrome wherein,
a) the predominant bowel habit is constipation and/or
b) the predominant bowel habit is alternating diarrhoea and constipation and/or
c) the treatment is administered parenterally.

Said medicament may further be for the treatment of abdominal and visceral pain associated with functional gastrointestinal disorders as irritable bowel syndrome and functional dyspepsia.

Said medicament may further be for the treatment of functional dyspepsia, for the relief of the symptom(s)
a) sensation of fullness and/or
b) inability to finish a normal sized meal and/or
c) pain after food intake and/or
d) nausea and/or
e) vomiting and/or
f) bloating and/or
g) belching and/or
h) regurgitation and/or
i) epigastric pain and/or
j) feeling of distention and/or
k) exess flatus
and any combination of the above.

It is further contemplated that the composition according to the invention

The invention further relates to a method of treatment comprising administration of said composition.

### DESCRIPTION OF DRAWINGS

FIG. 1. Structure of the family of the PP-fold peptides

FIG. 2. Plasma concentration of PYY1-36

FIG. 3. Plasma concentration of PYY3-36

### SEQUENCE LISTING

SEQ ID 1: Human PYY

### DEFINITIONS

AA: See “Amino acid”.

Amino acid: Entity comprising an amino terminal part (NH₂) and a carboxy terminal part (COOH) separated by a central part comprising a carbon atom, or a chain of carbon atoms, comprising at least one side chain or functional group. NH₂ refers to the amino group present at the amino terminal end of an amino acid or peptide, and COOH refers to the carboxy group present at the carboxy terminal end of an amino acid or peptide. The generic term amino acid comprises both natural and non-natural amino acids. Natural amino acids of standard nomenclature as listed in J. Biol. Chem., 243:3552-59 (1969) and adopted in 37 C.F.R., section 1.822(b)(2) belong to the group of amino acids listed in Table 1 herein below. Non-natural amino acids are those not listed in Table 1. Examples of non-natural amino acids are those listed e.g. in 37 C.F.R. section 1.822(b) (4), all of which are incorporated herein by reference. Amino acid residues described herein can be in the “D” or “L” isomeric form.

TABLE 1
Natural amino acids and their respective codes.

Symbols

| 1-Letter | 3-Letter | Amino acid |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | praline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

Amino acid residue: the term “amino acid residue” is meant to encompass amino acids, either standard amino acids, non-standard amino acids or pseudo-amino acids, which have been reacted with at least one other species, such as 2, for example 3, such as more than 3 other species. In particular amino acid residues may comprise an acyl bond in place of a free carboxyl group and/or an amine-bond and/or amide bond in place of a free amine group. Furthermore, reacted amino acid residues may comprise an ester or thioester bond in place of an amide bond.

Antibody: Are immunoglobulin molecules and active portions of immunoglobulin-molecules. Antibodies are for example intact immunoglobulin molecules or fragments thereof retaining the immunologic activity.

Antigen: The molecule recognised by an antibody. Usually a peptide, polypeptide or a multimeric polypeptide. Antigens are preferably capable of eliciting an immune response.

Chimera: A molecule consisting of at least two parts not found together in nature. A chimeric peptide or protein is a peptide or protein constructed by the fusion of two peptides or proteins. A chimeric DNA molecule is a DNA molecule that encodes a chimeric protein.

Concentration equivalent: A concentration equivalent is an equivalent dosage being defined as the dosage of a compound having the same response (as evaluated e.g. from a dosage-response curve) in vitro and/or in vivo as a known compound.

Frequency: The number of occurrences of a certain event within a certain period of time (e.g. the number of occurrences per day or per week).

Individual: A living animal or human. In preferred embodiments, the subject is a mammal, including humans and non-human mammals such as dogs, cats, pigs, cows, sheep, goats, horses, rats, and mice. In the most preferred embodiment, the subject is a human.

Isolated: is used to describe any of the various secretagogues, polypeptides and nucleotides disclosed herein, that have been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified.

Ligand: A molecule, for example a peptide, capable of specific binding to one or more cognate receptors. An antigen is, for example, a ligand to its cognate antibodies.

Medical disorder: By the term “medical disorder” is meant any disease or syndrome having a detrimental effect on an individual's physical and/or mental health.

MTD: Maximum tolerated dose. The maximum tolerated dose of a substance in a given test period should not induce a) overt toxicity, such as appreciable death of cells or organ dysfunction; b) a material reduction in life span, except by cancer induction; c) 10% or greater retardation of body weight gain (in growing animals).

Parenteral: For the purpose of this document “parenteral” is defined as being outside the alimentary canal. Thus, the term “parenteral administration” of a compound encompasses e.g. subcutaneous, intramuscular, intravenous, intranasal, buccal, intradermal and transdermal administration, as well as inhalation of said compound.

Peptide: Plurality of covalently linked amino acid residues defining a sequence and linked by amide bonds. The term is used analogously with oligopeptide and polypeptide. The amino acids may be both natural amino acids and non-natural amino acids, including any combination thereof. The natural and/or non-natural amino acids may be linked by peptide bonds or by non-peptide bonds. The term peptide also embraces post-translational modifications introduced by chemical or enzyme-catalyzed reactions, as are known in the art.

PYY: Peptide YY. Herein PYY represents both PYY1-36 and PYY3-36, the full length and a truncated version of PYY, respectively.

Receptor: A receptor is a molecule, such as a protein, glycoprotein and the like, that can specifically (non-randomly) bind to another molecule

Recombinant DNA (rDNA) molecule: A DNA molecule produced by operatively linking two DNA segments. Thus, a recombinant DNA molecule is a hybrid DNA molecule comprising at least two nucleotide sequences not normally found together in nature.

T_{1/2}: The half-life is the time for the concentration of a compound to decrease 50%.

### DETAILED DESCRIPTION OF THE DRAWINGS

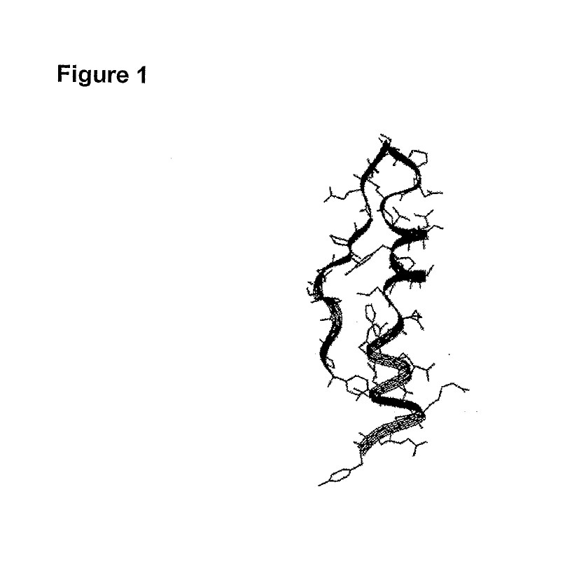

### FIG. 1.

### Structure of the Family of the PP-Fold Peptides

NPY, PYY, and PP share a common hairpin-like three-dimensional structure called the PP-fold (Fuhlendorf, J. et al. (1990) J. Biol. Chem.). All four peptides are 36 amino acids long with an amidated carboxy-terminus. The general structure of the PP-fold peptides has been established using x-ray crystallography of avian PP and confirmed in several studies using nuclear magnetic resonance (Keire D. A. et al. (2000) Biochemistry). Amino acid residues 1-8 form a type II proline helix followed by a loop. Residues 15-32 form an α-helix, and the four most carboxy-terminal residues are in a flexible loop conformation. NPY is one of the most evolutionary conserved peptides known. Despite a low degree of conservation in amino acid sequence between PP from different species as well as between PP and PYY and NPY, the general three-dimensional structure seems to be conserved in all PP-fold peptides.

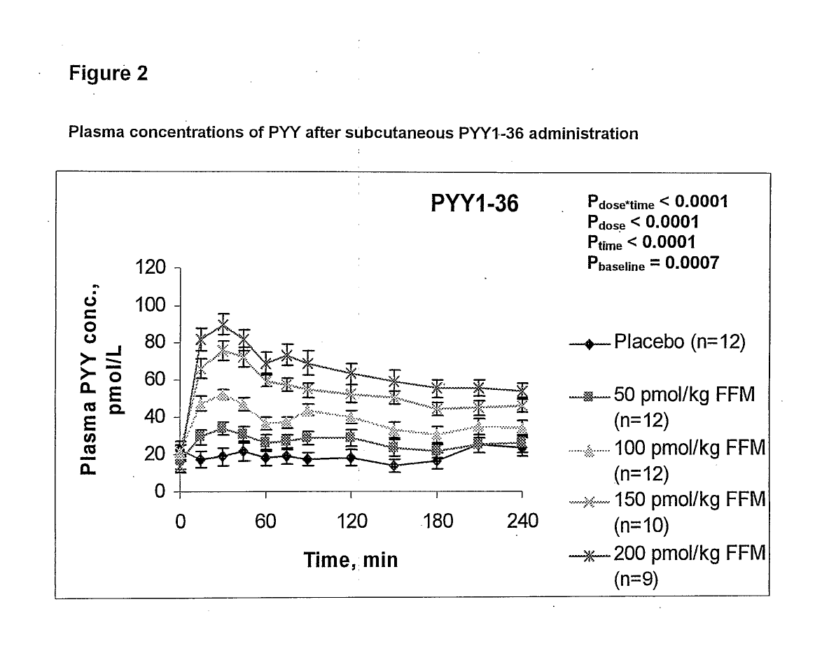

### FIG. 2

### PYY Plasma Concentration in Response to Subcutaneous PYY1-36 Administration.

PYY1-36 was administered to subjects as described in example 3. The plasma level of PYY was measured during a 4 hour time period following injection. The plasma level of PYY increases with in 15 minutes after administration.

A plasma level of 80-100 pmol/l is obtained using a dosage of 200 pmol/kg FFM.

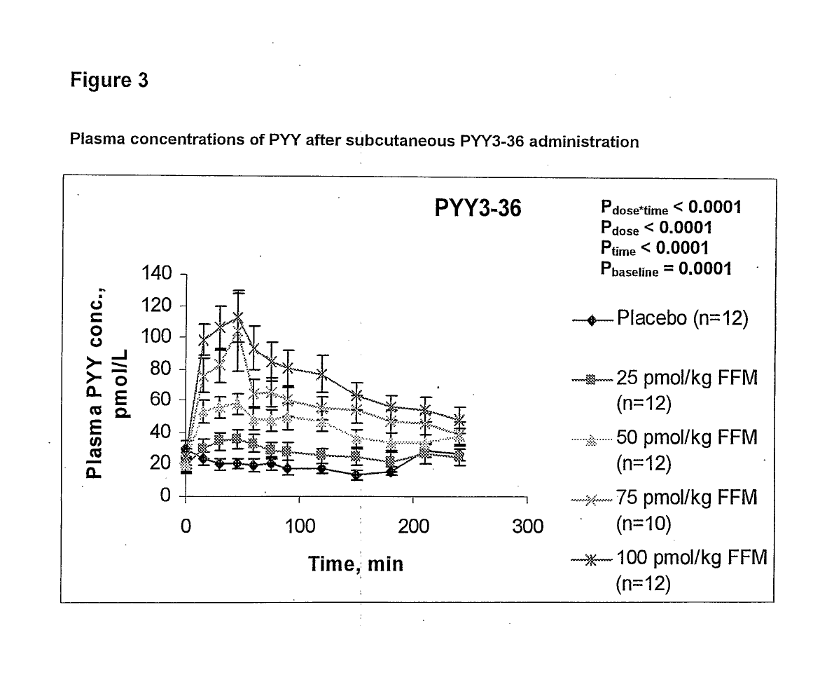

### FIG. 3

### PYY Plasma Concentration in Response to Subcutaneous PYY3-36 Administration.

PYY3-36 was administered to subjects as described in example 3. The plasma level of PYY was measured during a 4 hour time period following injection. The plasma level of PYY increases with in 15 minutes after administration.

A plasma level of 100-120 pmol/l is obtained using a dosage of 100 pmol/kg FFM.

### DETAILED DESCRIPTION OF THE INVENTION

### Gastrointestinal Disorders

The gastrointestinal disorders described in the background section of the application are characterised by the lack of known aetiology, and are subsequently diagnosed based on patients report of symptoms. The prevalent symptoms are abdominal discomfort and abdominal pain that may be accompanied by irregular stool frequency.

The symptoms of the patient may include; constipation, diarrhoea, abdominal pain, sensing of fullness, inability to finish a normal sized meal, bloating, vomiting, nausea, and epigastric pain.

### Individual in Need

According to the invention, any individual who may draw benefit from the compositions of the present invention may be treated with said compositions. Thus an individual who would draw benefit of the treatment are considered an individual in need of treatment.

Preferably, said individual is suffering from a gastrointestinal disorder. An individual in need may suffer from a disorder such as any of the above mentioned. An individual in need may suffer from any of the symptoms of the above mentioned disorders, such as abdominal pain, visceral pain, abdominal discomfort, diarrhoea, constipation, nausea, vomiting, bloating, belching or feeling of distension.

An individual in need may be an individual in risk of acquiring any of the above mentioned disorders.

As described above the symptoms associated with such gastrointestinal disorders have been difficult to treat. Surprisingly, the present invention discloses successful use of PYY and functional equivalents thereof for the treatment of gastrointestinal disorders.

### PYY

Throughout this document, peptide YY (PYY) is used as a general term covering PYY1-36 and PYY3-36. PYY1-36 is a 36 AA polypeptide with C- as well as N-terminal tyrosine amino acid residues. The polypeptide is produced by cleavage of a pre-polypeptide and is furthermore cleaved by depeptidyl peptidase IV yielding PYY3-36, as described above. Surprisingly, PYY has subsequently been found to be capable of suppressing symptoms of several gastrointestinal disorders such as IBS and functional dyspepsia.

### Functional Equivalents

Human PYY1-36 is identified by SEQ ID NO: 1. PYY3-36 is a truncated form of PYY where the two most N-terminal residues are deleted. Functional equivalents of PYY include PYY molecules originating from different species, such as mouse, rat, monkey, swine, bovine or other mammalian species. A functional equivalent may also be a homologue to PYY.

The invention relates to a composition comprising. PYY or a functional equivalent thereof, for the preparation of a pharmaceutical composition for the treatment of an gastrointestinal disorder with unknown aetiology.

In an embodiment of the invention the composition is for the preparation of a pharmaceutical composition for the treatment a gastrointestinal disorder with unknown aetiology characterized by the symptoms
a) constipation and/or
b) diarrhoea and/or
c) abdominal pain

In one embodiment the composition comprising PYY or a functional equivalent thereof is for the preparation of a pharmaceutical composition for the treatment of irritable bowel syndrome wherein
a) the predominant bowel habit is constipation and/or
b) the predominant bowel habit is alternating diarrhoea and constipation and/or
c) the treatment is administered parenterally.

In a further embodiment the composition is for the treatment of functional dyspepsia.

In a further embodiment the composition is for the treatment of abdominal pain, visceral pain, such as pain in the left upper quadrant, such as pain in the right upper quadrant, such as pain in the left lower quadrant, such as pain in the right lower quadrant.

In an embodiment the composition is for the treatment of

In an embodiment the composition is the treatment of pain associated with either IBS or functional dyspepsia.

In an embodiment the composition is for the relief of the symptom(s)
l) sensation of fullness and/or
m) inability to finish a normal sized meal and/or
n) pain after food intake and/or
o) nausea and/or
p) vomiting and/or
q) bloating and/or
r) belching and/or
s) regurgitation and/or
t) epigastric pain and/or
u) feeling of distention and/or
v) exess flatus
and any combinations hereof.

In a preferred embodiment the composition comprises human PYY. In a more preferred embodiment the composition comprises human PYY1-36 as defined by SEQ ID NO 1. In a further preferred embodiment the composition comprises human PYY3-36 as defined by amino acid 3-3 in SEQ ID NO. 1.

### Homologues

A homologue shall be construed as a molecule that shares some identity to the molecule, here PYY represented by both PYY1-36 and PYY3-36. The homology may be expressed as the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N- or C-terminal extensions nor insertions shall be construed as reducing identity or homology. Methods and computer programs for the alignment are well known in the art. Sequence identity may be measured using sequence analysis software (e.g., Sequence Analysis Software Package, Genetics Computer Group, University of Wisconsin Biotechnology Centre, 1710 University Ave., Madison, Wis. 53705). This software matches similar sequences by assigning degrees of homology to various substitutions, deletions, and other modifications.

A homologue of one or more of the sequences specified herein may vary in one or more amino acids as compared to the sequences defined, but is capable of performing the same function, i.e. a homologue may be envisaged as a “functional equivalent” of a predetermined sequence.

As described above a functional equivalent of any of the predetermined sequences herein may be defined as:
i) homologues comprising an amino acid sequence capable of being recognised by an antibody, said antibody also recognising PYY (PYY1-36 and/or PYY3-36), and/or
ii) homologues comprising an amino acid sequence capable of binding selectively to an NPY receptor (for example measured as described in example 1), and/or
iii) homologues having a substantially similar or higher binding affinity to NPY receptors than PYY (PYY1-36 or PYY3-36) and with a binding affinity preferably high than 100 nM and/or
iv) homologues with at least 60% identity to human PYY identified by SEQ ID NO 1 and/or
v) homologues consisting of fragments of human PYY identified by SEQ ID NO 1, wherein the fragments comprise a stretch of at least 6 continuous amino acids of SEQ ID NO 1.

Human PYY1-36 has the sequence shown in SEQ ID NO: 1. Human PYY3-36 is 34 amino acids long and has the sequence shown in SEQ ID NO: 1 except for the deletion of the two N-terminal amino acids.

Examples of homologues comprise one or more conservative amino acid substitutions including one or more conservative amino acid substitutions within the same group of predetermined amino acids, or a plurality of conservative amino acid substitutions, wherein each conservative substitution is generated by substitution within a different group of predetermined amino acids.

Homologues may thus comprise conservative substitutions independently of one another, wherein at least one glycine (Gly) of said homologue is substituted with an amino acid selected from the group of amino acids consisting of Ala, Val, Leu, and Ile, and independently thereof, homologues, wherein at least one of said alanines (Ala) of said homologue thereof is substituted with an amino acid selected from the group of amino acids consisting of Gly, Val, Leu, and Ile, and independently thereof, homologues, wherein at least one valine (Val) of said homologue thereof is substituted with an amino acid selected from the group of amino acids consisting of Gly, Ala, Leu, and Ile, and independently thereof, homologues thereof, wherein at least one of said leucines (Leu) of said homologue thereof is substituted with an amino acid selected from the group of amino acids consisting of Gly, Ala, Val, and Ile, and independently thereof, homologues thereof, wherein at least one isoleucine (Ile) of said homologues thereof is substituted with an amino acid selected from the group of amino acids consisting of Gly, Ala, Val and Leu, and independently thereof, homologues thereof wherein at least one of said aspartic acids (Asp) of said homologue thereof is substituted with an amino acid selected from the group of amino acids consisting of Glu, Asn, and Gln, and independently thereof, homologues thereof, wherein at least one of said phenylalanines (Phe) of said homologues thereof is substituted with an amino acid selected from the group of amino acids consisting of Tyr, Trp, His, Pro, and preferably selected from the group of amino acids consisting of Tyr and Trp, and independently thereof, homologues thereof, wherein at least one of said tyrosines (Tyr) of said homologues thereof is substituted with an amino acid selected from the group of amino acids consisting of Phe, Trp, His, Pro, preferably an amino acid selected from the group of amino acids consisting of Phe and Trp, and independently thereof, homologues thereof, wherein at least one of said arginines (Arg) of said fragment is substituted with an amino acid selected from the group of amino acids consisting of Lys and His, and independently thereof, homologues thereof, wherein at least one lysine (Lys) of said homologues thereof is substituted with an amino acid selected from the group of amino acids consisting of Arg and His, and independently thereof, homologues thereof, wherein at least one of said aspargines (Asn) of said homologues thereof is substituted with an amino acid selected from the group of amino acids consisting of Asp, Glu, and Gln, and independently thereof, homologues thereof, wherein at least one glutamine (Gln) of said homologues thereof is substituted with an amino acid selected from the group of amino acids consisting of Asp, Glu, and Asn, and independently thereof, homologues thereof, wherein at least one proline (Pro) of said homologues thereof is substituted with an amino acid selected from the group of amino acids consisting of Phe, Tyr, Trp, and His, and independently thereof, homologues thereof, wherein at least one of said cysteines (Cys) of said homologues thereof is substituted with an amino acid selected from the group of amino acids consisting of Asp, Glu, Lys, Arg, His, Asn, Gln, Ser, Thr, and Tyr.

Conservative substitutions may be introduced in any position of a preferred predetermined sequence. It may however also be desirable to introduce non-conservative substitutions, particularly, but not limited to, a non-conservative substitution in any one or more positions.

A non-conservative substitution leading to the formation of a functionally equivalent homologue of the sequences herein would for example i) differ substantially in polarity, for example a residue with a non-polar side chain (Ala, Leu, Pro, Trp, Val, Ile, Leu, Phe or Met) substituted for a residue with a polar side chain such as Gly, Ser, Thr, Cys, Tyr, Asn, or Gln or a charged amino acid such as Asp, Glu, Arg, or Lys, or substituting a charged or a polar residue for a non-polar one; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Substitution of amino acids may in one embodiment be made based upon their hydrophobicity and hydrophilicity values and the relative similarity of the amino acid side-chain substitutions, including charge, size, and the like. Exemplary amino acid substitutions which take one of the foregoing characteristics into consideration are well known to those of skill in the art and include: arginine and lysine; glutamate and aspartate; serine and threonine; glutamine and asparagine; and valine, leucine and isoleucine.

In a preferred embodiment the homologue has an amino acid sequence at least 60% identical to SEQ ID NO 1.

More preferably the identity is at least 65%, such as at least 70% identical, such as at least 75% identical, such as at least 80% identical, such as at least 85% identical, such as at least 90% identical, such as at least 95% identical, such as at least 98% identical to SEQ ID NO 1.

In a preferred embodiment the functional equivalent comprise the amino acids corresponding to the 6 N-terminal amino acids of PYY1-36 as defined in SEQ ID NO:1 (Tyr Pro Ile Lys Pro Glu). The functional equivalent may comprise 8 N-terminal amino acids of PYY1-36 as defined in SEQ ID (Tyr Pro Ile Lys Pro Glu Ala Pro), or such as 10 N-terminal amino acids of PYY1-36 as defined in SEQ ID (Tyr Pro Ile Lys Pro Glu Ala Pro Gly Glu) or such as 12 N-terminal amino acids of PYY1-36 as defined in SEQ ID (Tyr Pro Ile Lys Pro Glu Ala Pro Gly Glu Asp Ala), or such as 14 N-terminal amino acids of PYY1-36 as defined in SEQ ID (Tyr Pro Ile Lys Pro Glu Ala Pro Gly Glu Asp Ala Ser Pro)), or such as 16 N-terminal amino acids of PYY1-36 as defined in SEQ ID (Tyr Pro Ile Lys Pro Glu Ala Pro Gly Glu Asp Ala Ser Pro Glu Glu), or such as 18 N-terminal amino acids of PYY1-36 as defined in SEQ ID (Tyr Pro Ile Lys Pro Glu Ala Pro Gly Glu Asp Ala Ser Pro Glu Glu Leu Asn), or such as 20 N-terminal amino acids of PYY1-36 as defined in SEQ ID (Tyr Pro Ile Lys Pro Glu Ala Pro Gly Glu Asp Ala Ser Pro Glu Glu Leu Asn Arg Tyr), or such as 22 N-terminal amino acids of PYY1-36 as defined in SEQ ID (Tyr Pro Ile Lys Pro Glu Ala Pro Gly Glu Asp Ala Ser Pro Glu Glu Leu Asn Arg Tyr Tyr Ala), or such as 24 N-terminal amino acids of PYY1-36 as defined in SEQ ID (Tyr Pro Ile Lys Pro Glu Ala Pro Gly Glu Asp Ala Ser Pro Glu Glu Leu Asn Arg Tyr Tyr Ala Ser Leu), or such as 26 N-terminal amino acids of PYY1-36 as defined in SEQ ID (Tyr Pro Ile Lys Pro Glu Ala Pro Gly Glu Asp Ala Ser Pro Glu Glu Leu Asn Arg Tyr Tyr Ala Ser Leu Arg His), or such as 28 N-terminal amino acids of PYY1-36 as defined in SEQ ID (Tyr Pro Ile Lys Pro Glu Ala Pro Gly Glu Asp Ala Ser Pro Glu Glu Leu Asn Arg Tyr Tyr Ala Ser Leu Arg His Tyr Leu), or such as 30 N-terminal amino acids of PYY1-36 as defined in SEQ ID (Tyr Pro Ile Lys Pro Glu Ala Pro Gly Glu Asp Ala Ser Pro Glu Glu Leu Asn Arg Tyr Tyr Ala Ser Leu Arg His Tyr Leu Asn Leu), or such as 32 N-terminal amino acids of PYY1-36 as defined in SEQ ID (Tyr Pro Ile Lys Pro Glu Ala Pro Gly Glu Asp Ala Ser Pro Glu Glu Leu Asn Arg Tyr Tyr Ala Ser Leu Arg His Tyr Leu Asn Leu Val Thr), or such as 34N-terminal amino acids of PYY1-36 as defined in SEQ ID (Tyr Pro Ile Lys Pro Glu Ala Pro Gly Glu Asp Ala Ser Pro Glu Glu Leu Asn Arg Tyr Tyr Ala Ser Leu Arg His Tyr Leu Asn Leu Val Thr Arg Gln).

In a further preferred embodiment the functional equivalent comprise the amino acids corresponding to the 6 N-terminal amino acids of PYY3-36 as defined in SEQ ID NO:1 (Ile Lys Pro Glu Ala Pro), or such as 8 N-terminal amino acids of PYY3-36 as defined in SEQ ID (Ile Lys Pro Glu Ala Pro Gly Glu) or such as 10 N-terminal amino acids of PYY3-36 as defined in SEQ ID (Ile Lys Pro Glu Ala Pro Gly Glu Asp Ala), or such as 12 N-terminal amino acids of PYY3-36 as defined in SEQ ID (Ile Lys Pro Glu Ala Pro Gly Glu Asp Ala Ser Pro)), or such as 14 N-terminal amino acids of PYY3-36 as defined in SEQ ID (Ile Lys Pro Glu Ala Pro Gly Glu Asp Ala Ser Pro Glu Glu), or such as 16 N-terminal amino acids of PYY3-36 as defined in SEQ ID (Ile Lys Pro Glu Ala Pro Gly Glu Asp Ala Ser Pro Glu Glu Leu Asn), or such as 18 N-terminal amino acids of PYY3-36 as defined in SEQ ID (Ile Lys Pro Glu Ala Pro Gly Glu Asp Ala Ser Pro Glu Glu Leu Asn Arg Tyr), or such as 20 N-terminal amino acids of PYY3-36 as defined in SEQ ID (Ile Lys Pro Glu Ala Pro Gly Glu Asp Ala Ser Pro Glu Glu Leu Asn Arg Tyr Tyr Ala), or such as 22 N-terminal amino acids of PYY3-36 as defined in SEQ ID (Ile Lys Pro Glu Ala Pro Gly Glu Asp Ala Ser Pro Glu Glu Leu Asn Arg Tyr Tyr Ala Ser Leu), or such as 24 N-terminal amino acids of PYY3-36 as defined in SEQ ID (Tyr Pro Ile Lys Pro Glu Ala Pro Gly Glu Asp Ala Ser Pro Glu Glu Leu Asn Arg Tyr Tyr Ala Ser Leu Arg His), or such as 26 N-terminal amino acids of PYY3-36 as defined in SEQ ID (Ile Lys Pro Glu Ala Pro Gly Glu Asp Ala Ser Pro Glu Glu Leu Asn Arg Tyr Tyr Ala Ser Leu Arg His Tyr Leu), or such as 28 N-terminal amino acids of PYY3-36 as defined in SEQ ID (Ile Lys Pro Glu Ala Pro Gly Glu Asp Ala Ser Pro Glu Glu Leu Asn Arg Tyr Tyr Ala Ser Leu Arg His Tyr Leu Asn Leu), or such as 30 N-terminal amino acids of PYY3-36 as defined in SEQ ID (Ile Lys Pro Glu Ala Pro Gly Glu Asp Ala Ser Pro Glu Glu Leu Asn Arg Tyr Tyr Ala Ser Leu Arg His Tyr Leu Asn Leu Val Thr), or such as 32 N-terminal amino acids of PYY3-36 as defined in SEQ ID (Ile Lys Pro Glu Ala Pro Gly Glu Asp Ala Ser Pro Glu Glu Leu Asn Arg Tyr Tyr Ala Ser Leu Arg His Tyr Leu Asn Leu Val Thr Arg Gln).

In a preferred embodiment the functional equivalent comprise the amino acids corresponding to the 6 C-terminal amino acids of PYY1-36 as defined in SEQ ID NO 1 (Val Thr Arg Gln Arg Tyr), or such as the 8 C-terminal amino acids of PYY1-36 as defined in SEQ ID NO 1 (Asn Leu Val Thr Arg Gln Arg Tyr), or such as the 10 C-terminal amino acids of PYY1-36 as defined in SEQ ID NO 1 (Tyr Leu Asn Leu Val Thr Arg Gln Arg Tyr), or such as the 12 C-terminal amino acids of PYY1-36 as defined in SEQ ID NO 1 (Arg His Tyr Leu Asn Leu Val Thr Arg Gln Arg Tyr), or such as the 14 C-terminal amino acids of PYY1-36 as defined in SEQ ID NO 1 (Ser Leu Arg His Tyr Leu Asn Leu Val Thr Arg Gln Arg Tyr), or such as the 16 C-terminal amino acids of PYY1-36 as defined in SEQ ID NO 1 (Ser Leu Arg His Tyr Leu Asn Leu Val Thr Arg Gln Arg Tyr Tyr Ala), or such as the 18 C-terminal amino acids of PYY1-36 as defined in SEQ ID NO 1 (Arg Tyr Ser Leu Arg His Tyr Leu Asn Leu Val Thr Arg Gln Arg Tyr Tyr Ala), or such as the 20 C-terminal amino acids of PYY1-36 as defined in SEQ ID NO 1 (Leu Asn Arg Tyr Ser Leu Arg His Tyr Leu Asn Leu Val Thr Arg Gln Arg Tyr Tyr Ala), or such as the 22 C-terminal amino acids of PYY1-36 as defined in SEQ ID NO 1 (Glu Glu Leu Asn Arg Tyr Ser Leu Arg His Tyr Leu Asn Leu Val Thr Arg Gln Arg Tyr Tyr Ala), or such as the 24 C-terminal amino acids of PYY1-36 as defined in SEQ ID NO 1 (Ser Pro Glu Glu Leu Asn Arg Tyr Ser Leu Arg His Tyr Leu Asn Leu Val Thr Arg Gln Arg Tyr Tyr Ala), or such as the 26 C-terminal amino acids of PYY1-36 as defined in SEQ ID NO 1 (Asp Ala Ser Pro Glu Glu Leu Asn Arg Tyr Ser Leu Arg His Tyr Leu Asn Leu Val Thr Arg Gln Arg Tyr Tyr Ala), or such as the 28 C-terminal amino acids of PYY1-36 as defined in SEQ ID NO 1 (Gly Glu Asp Ala Ser Pro Glu Glu Leu Asn Arg Tyr Ser Leu Arg His Tyr Leu Asn Leu Val Thr Arg Gln Arg Tyr Tyr Ala), or such as the 30 C-terminal amino acids of PYY1-36 as defined in SEQ ID NO 1 (Ala Pro Gly Glu Asp Ala Ser Pro Glu Glu Leu Asn Arg Tyr Ser Leu Arg His Tyr Leu Asn Leu Val Thr Arg Gln Arg Tyr Tyr Ala), or such as the 32 C-terminal amino acids of PYY1-36 as defined in SEQ ID NO 1 (Pro Glu Ala Pro Gly Glu Asp Ala Ser Pro Glu Glu Leu Asn Arg Tyr Ser Leu Arg His Tyr Leu Asn Leu Val Thr Arg Gln Arg Tyr Tyr Ala), or such as the 34 C-terminal amino acids of PYY1-36 as defined in SEQ ID NO 1 (Tyr Ala Ile Lys Pro Glu Ala Pro Gly Glu Asp Ala Ser Pro Glu Glu Leu Asn Arg Tyr Ser Leu Arg His Tyr Leu Asn Leu Val Thr Arg Gln Arg Tyr).

In another preferred embodiment the functional equivalent may comprise internal amino acids of PYY1-36 such as amino acid 16-21 of PYY 1-36 as defined in sequence ID NO 1 (Glu Leu Asn Arg Tyr Tyr), or such as amino acid 15-22 of PYY 1-36 as defined in sequence ID NO 1 (Glu Glu Leu Asn Arg Tyr Tyr Ala), or such as amino acid 14-23 of PYY 1-36 as defined in sequence ID NO 1 (Pro Glu Glu Leu Asn Arg Tyr Tyr Ala Ser), or such as amino acid 13-24 of PYY 1-36 as defined in sequence ID NO 1 (Ser Pro Glu Glu Leu Asn Arg Tyr Tyr Ala Ser Leu), or such as amino acid 12-25 of PYY 1-36 as defined in sequence ID NO 1 (Ala Ser Pro Glu Glu Leu Asn Arg Tyr Tyr Ala Ser Leu Arg), or such as amino acid 11-26 of PYY 1-36 as defined in sequence ID NO 1 (Asp Ala Ser Pro Glu Glu Leu Asn Arg Tyr Tyr Ala Ser Leu Arg His), or such as amino acid 10-27 of PYY 1-36 as defined in sequence ID NO 1 (Glu Asp Ala Ser Pro Glu Glu Leu Asn Arg Tyr Tyr Ala Ser Leu Arg His Tyr), or such as amino acid 9-28 of PYY 1-36 as defined in sequence ID NO 1 (Gly Glu Asp Ala Ser Pro Glu Glu Leu Asn Arg Tyr Tyr Ala Ser Leu Arg His Tyr Leu), or such as amino acid 8-29 of PYY 1-36 as defined in sequence ID NO 1 (Pro Gly Glu Asp Ala Ser Pro Glu Glu Leu Asn Arg Tyr Tyr Ala Ser Leu Arg His Tyr Leu Asn), or such as amino acid 7-30 of PYY 1-36 as defined in sequence ID NO 1 (Ala Pro Gly Glu Asp Ala Ser Pro Glu Glu Leu Asn Arg Tyr Tyr Ala Ser Leu Arg His Tyr Leu Asn Leu), or such as amino acid 6-31 of PYY 1-36 as defined in sequence ID NO 1 (Glu Ala Pro Gly Glu Asp Ala Ser Pro Glu Glu Leu Asn Arg Tyr Tyr Ala Ser Leu Arg His Tyr Leu Asn Leu Val), or such as amino acid 5-32 of PYY 1-36 as defined in sequence ID NO 1 (Pro Glu Ala Pro Gly Glu Asp Ala Ser Pro Glu Glu Leu Asn Arg Tyr Tyr Ala Ser Leu Arg His Tyr Leu Asn Leu Val Thr), or such as amino acid 4-33 of PYY 1-36 as defined in sequence ID NO 1 (Lys Pro Glu Ala Pro Gly Glu Asp Ala Ser Pro Glu Glu Leu Asn Arg Tyr Tyr Ala Ser Leu Arg His Tyr Leu Asn Leu Val Thr Arg), or such as amino acid 3-34 of PYY 1-36 as defined in sequence ID NO 1 (Ile Lys Pro Glu Ala Pro Gly Glu Asp Ala Ser Pro Glu Glu Leu Asn Arg Tyr Tyr Ala Ser Leu Arg His Tyr Leu Asn Leu Val Thr Arg Gln), or such as amino acid 2-35 of PYY 1-36 as defined in sequence ID NO 1 (Pro Ile Lys Pro Glu Ala Pro Gly Glu Asp Ala Ser Pro Glu Glu Leu Asn Arg Tyr Tyr Ala Ser Leu Arg His Tyr Leu Asn Leu Val Thr Arg Gln Arg) or such as amino acid 2-36 of PYY 1-36 as defined in sequence ID NO 1 (Pro Ile Lys Pro Glu Ala Pro Gly Glu Asp Ala Ser Pro Glu Glu Leu Asn Arg Tyr Tyr Ala Ser Leu Arg His Tyr Leu Asn Leu Val Thr Arg Gln Arg Tyr), or such as amino acid 4-36 of PYY 1-36 as defined in sequence ID NO 1 (Lys Pro Glu Ala Pro Gly Glu Asp Ala Ser Pro Glu Glu Leu Asn Arg Tyr Tyr Ala Ser Leu Arg His Tyr Leu Asn Leu Val Thr Arg Gln Arg Tyr).

In an embodiment the functional equivalent comprise any of the above sequences with conservative amino acid substitutions, such as one substitution, or such as two substitutions, or such as two substitutions, or such as more than two substitutions, or such as more than four substitutions.

Further included are functional equivalent know from the literature, such as the PYY agonists described in WO 03/057235 and references therein.

### Covalent Modifications

The functional equivalent may comprise any type of modifications. Nearly 200 structurally distinct covalent modifications have been identified thus far, ranging in size and complexity from conversion of amides to carboxylic acids, to the attachment of multiple complex oligosaccharides. Such modifications include phosphorylation, acetylation, ubiquination, lipidation (acetylation, prenylation, farnesylation, geranylation, palmitoylation, myristoylation), methylation, carboxylation, sulfunation and O- or N-glycosylations.

A subset of modifications is dependent on vitamin C as a cofactor. This include proline and lysine hydroxylations and carboxy terminal amidation.

In an embodiment PYY or the functional equivalent comprise a C-terminal amidation. In a preferred embodiment the C-terminal tyrosine residue of PYY or a functional equivalent is amidated.

### Protecting Group

The functional equivalent may according to the invention comprise protecting group at the N-terminus or the C-terminus or at both.

A protecting group covalently joined to the N-terminal amino group reduces the reactivity of the amino terminus under in vivo conditions. Amino protecting groups include —C1-10 alkyl, —C1-10 substituted alkyl, —C2-10 alkenyl, —C2-10 substituted alkenyl, aryl, —C1-6 alkyl aryl, —C(O)— (CH2) 1-6-COOH, —C(O)—C1-6 alkyl, —C(O)-aryl, —C(O)—O—C1-6 alkyl, or —C(O)—O-aryl. Preferably, the amino terminus protecting group is acetyl, propyl, succinyl, benzyl, benzyloxycarbonyl or tbutyloxycarbonyl.

A protecting group covalently joined to the C-terminal carboxy group reduces the reactivity of the carboxy terminus under in vivo conditions. The carboxy terminus protecting group is preferably attached to the a-carbonyl group of the last amino acid. Carboxy terminus protecting groups include amide, methylamide, and ethylamide.

### Conjugates

PYY or the functional equivalent of PYY may conjugated to another entity, in order for example, to prolong its half-life. Conjugation can improve the delivery of targeted doses, prevent breakdown, and increase bioavailability in circulation. The conjugate may be any molecule.

The preparation of conjugates is well known in the art se for example Hermanson G T. Bioconjugate Techniques. New York: Academic Press; 1996, Aslam M, Dent A H. Bioconjugation: protein coupling techniques for the biomedical sciences. Houndsmills, England: Macmillan Publishers; 1999, and Wong S S. Chemistry of protein conjugation and crosslinking. Boca Raton, Fla.: CRC Press; 1991.

Most methods use amine-reactive reagents or thiol-reactive reagent. In the preparation of conjugates advantages may be achieved through the use of certain linkers. For example, linkers that contain a disulfide bond that is sterically “hindered” are often preferred, due to their greater stability in vivo, thus preventing release of the toxin moiety prior to binding at the site of action. It is generally desired to have a conjugate that will remain intact under conditions found everywhere in the body except the intended site of action, at which point it is desirable that the conjugate have good “release” characteristics.

Different conjugates have been described, for example, use of the A chain of ricin is described in U.S. Pat. No. 4,340,535 incorporated herein by reference. Examples of peptide conjugates based on Ac-RYY(RK)(WI)RK)—NH₂ (where the brackets show allowable variation of amino acid residues) may be found in US patent application 2003040472.

In an embodiment of the invention PYY or the functional equivalent is conjugated to another entity.

The molecules may be conjugated as described above, or by peptide bonds, before or after synthesis and purification. The fusion may be obtained by any suitable methods, for example, but not exclusively, by recombinant DNA technology. In a preferred embodiment the fusion of is made by recombinant DNA technology, such as a fusion of the nucleotide sequence encoding the binding member and the nucleotide sequence encoding the ligand is made and the fusion is thereby encoded by a single nucleotide sequence. The fusion polypeptide may be expressed and purified as a single polypeptide molecule, using any suitable method, as described for the purification of a binding member. The fusion polypeptide may include insertion of a linker, such as a peptide of at least 2 AA, such as at least 5 AA, such as at least 8 AA, such as at least 15 AA, such as at least 20 AA

In one embodiment PYY or a functional equivalent there of is conjugated with another entity using a linker of at least 2AA.

### Methods for Production of PYY

PYY or a functional equivalent thereof can be produced using techniques well known in the art. For example, a polypeptide region of a PYY can be chemically or biochemically synthesized and modified. Techniques for chemical synthesis of polypeptides are well known in the art such as solid phase peptide synthesis (see e.g., Vincent in Peptide and Protein Drug Delivery, New York, N.Y., Dekker, 1990.) Examples of techniques for biochemical synthesis involving the introduction of a nucleic acid into a cell and expression of nucleic acids are provided in Ausubel, Current Protocols in Molecular Biology, John Wiley, 1987-1998, and Sambrook et al., in Molecular Cloning, A Laboratory Manual, 2 d Edition, Cold Spring Harbor Laboratory Press, 1989.

PYY may according to the invention by synthesised by solid phase peptide synthesis (se example 1).

An second example of how PYY according to the invention is produced is described in brief below. The human PYY of the present invention may be produced by the following process:
(a) constructing, by conventional techniques, an expression vector containing an operon with a DNA sequence encoding human PYY or a functional equivalent thereof, thereby producing the vector of the invention;
(b) transfecting the expression vectors into a host cell by conventional techniques to produce the transfected host cell of the invention; and
(c) culturing the transfected cell by conventional techniques to produce the human PYY of the invention.

The host cell may be cotransfected with a second vector for optimization of the production process. The two vectors may contain different selectable markers. The coding sequences of PYY may comprise cDNA or genomic DNA or both.

The host cell used to express the PYY of the invention may be either a bacterial cell such as Escherichia coli, or a eukaryotic cell, such as S. cerevisiae or P. pastoris. In particular a mammalian cell line may be used, such as Hela, CHO or any other suitable host cell known by the person skilled in the art.

The general methods by which the vectors of the invention may be constructed, transfection methods required to produce the host cell of the invention and culture methods required to produce the polypeptide of the invention from such host cells are all conventional techniques. Likewise, once produced, the polypeptide of the invention may be purified according to standard procedures as described below.

### Purification of PYY

After production, PYY or a functional equivalent thereof is preferably purified. The method of purification used is dependent upon several factors, including the purity required, the source of PYY or a functional equivalent, the intended use and the species in which PYY or a functional equivalent was produced.

Any suitable conventional methods of purifying polypeptides including precipitation and column chromatography are well known to one of skill in the purification arts, including cross-flow filtration, ammonium sulphate precipitation, affinity column chromatography, gel electrophoresis and the like may be used.

### PYY Composition

According to the invention, a composition comprising PYY or a functional equivalent thereof may preferably be produced by chemical or biochemical synthesized or recombinant methods, and may preferably be free of any contaminants present in blood such as infectious agents.

In a preferred embodiment the PYY composition have a concentration of at least 10 nM such as at least 50 nM, such as at least 0.2 μM, such as at least 0.5 μM, such as at least 1 μM, such as at least 2 μM, such as at least 5 μM, such as at least 10 μM, such as at least 20 μM, such as at least 50 μM, such as at least 0.2 mM, such as at least 0.5 μM, such as at least 1 mM, such as at least 2 mM, such as at least 5 mM of PYY or a functional equivalent thereof.

The PYY composition may be stored as a dry composition, for example lyophilized (freeze-dried) or spray dried to improve the stability of PYY. Such compositions are reconstituted with liquid solutions prior to use. Generally, the protein concentration of the reconstituted formulation is about 2-40 times greater than the protein concentration in the mixture before lyophilization; thus this allows the production of a PYY composition of a high concentration. When reconstituted with a diluent comprising a preservative (such as bacteriostatic water for injection, BWFI), the reconstituted formulation may be used as a multi-dose formulation. Such a formulation is useful, for example, where the patient requires frequent subcutaneous administrations.

In another embodiment the PYY composition may be a liquid composition of high stability. The PYY composition may be meant for mixing with a suitable diluent prior to use.

The PYY composition may further comprise pharmaceutically acceptable salts, as well as pharmaceutically acceptable carriers and diluents.

### Pharmaceutical Compositions

Pharmaceutical compositions of the present invention may be prepared by conventional techniques, e.g. as described in Remington: The Science and Practice of Pharmacy 1995, edited by E. W. Martin, Mack Publishing Company, 19th edition, Easton, Pa. The compositions may appear in conventional forms, for example solutions or suspensions.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon an individual without the production of undesirable physiological effects such as nausea, dizziness, gastric upset and the like.

According to the present invention, the pharmaceutical composition may comprise PYY or a functional equivalent thereof and pharmaceutical acceptable salts.

The pharmaceutical composition according to the present invention further preferably comprises pharmaceutically acceptable salts, a pharmaceutically acceptable carrier and/or a diluent. The pharmaceutical composition may further comprise vehicles, excipients and/or transport molecules.

The pharmaceutical composition may be produced prior to use by mixing a PYY composition with an appropriate diluent.

The compositions of the present invention may preferably be administered to an individual in any way so as to achieve a beneficial effect, preferably for treatment of a gastrointestinal disorder.

An aspect of the invention relates to a method of treatment comprising administration of the pharmaceutical composition according to the invention.

The pharmaceutical composition according to the invention may be formulated for administered by any suitable route, such as peripherally, parenterally or orally.

The pharmaceutical composition according to the invention is preferably formulated for parenteral administration, such as via a subcutaneous, intramuscular, intravenous, intranasal, inhalation, buccal, intradermal and transdermal administration route. Further including formulation for administration via rectal suppositories.

### Second Active Ingredient

The patient suffering from an eating disorder event may benefit from additional treatments. This may e.g. involve anti-depressants, such as selective serotonin reuptake inhibitors (SSRIs), serotonin noradrenalin reuptake inhibitors (SNRIs), norepinephrine serotonin reuptake inhibitors (NSRIs), selective noradrenalin reuptake inhibitors, tetracyclic antidepressants, non-selective monoamine reuptake inhibitors including tricyclic antidepressants (TCAs), selective reversible monoamine reuptake inhibitors and antidepressants with other mechanisms of action, e.g. mirtazapin. Examples of SSRIs are citalopram, escitalopram, fluoxetine, fluvoxamine, paroxetine and sertraline. An example of an SNRI is venlafaxine. An example of an NSRI is milnacipran.

In an embodiment of the invention, the composition comprises a second active ingredient in addition to PYY or a functional equivalent thereof.

In another embodiment, an anti-emetic may be used as a second active ingredient. This is of particular interest in cases where the treatment with PYY or a functional equivalent thereof gives rise to nausea and/or emesis.

### Anti-Emetic Drugs

In the present context, an “anti-emetic” drug is one which counteracts (i.e. reduces or removes) nausea or emesis (vomiting). The experience of nausea and emesis may have many causes and the relief or reduction of the symptoms may be obtained by various mechanisms. The major groups of drugs useful for the treatment of nausea and emesis are; Neuroleptics/anti-psychotics, Antihistamines, Anticholinergic agents, Steroids (corticosteroids), 5HT3-receptor antagonists (serotonin receptor antagonist), NK1-receptor antagonists (Neurokinin 1 substance P receptor antagonists), Antidopaminergic agents/dopamine receptor antagonists, Benzodiazapines, Cannabinoids. Here below is a non-exhaustive list of members of the different groups of compounds.
1. Neuroleptics/anti-psychotics
   a. Dixyrazine
   b. Haloperidol
   c. Prochlorperazine (Compazine®)
2. Antihistamines
   a. piperazine derivatives
      i. cyclizine
      ii. meclizine
      iii. cinnarizine
   b. Promethazine
   c. Dimenhydrinate
   d. Diphenhydramine
   e. Hydroxyzine
   f. Buclizine
   g. Meclizine hydrochloride (Bonine, Antivert)
3. Anticholinergic agents (Inhibitors of the acetylcholine receptors.)
   a. Scopolamine
   b. Glycopyrron
   c. Hyoscine
      i. Artane (Trihexy-5 trihexyphenidyl hydrochloride)
      ii. Cogentin (benztropine mesylate)
      iii. Akineton (biperiden hydrochloride)
      iv. Disipal (Norflex orphenadrine citrate)
      v. Kemadrin (procyclidine hydrochloride)
4. Steroids (corticosteroids)
   a. Betamethasone
   b. Dexamethasone
   c. Methylprednisolone
   d. Prednisone®,
   e. Trimethobenzamide (Tigan)
5. 5HT3-receptor antagonists (serotonin receptor antagonist)
   a. Granisetron
   b. Dolasetron
   c. Ondansetron (hydrochloride)
   d. Tropisetron
   e. Ramosetron
   f. Palonosetron
   g. Alosetron
   h. Bemesetron
   i. Zatisetron
   j. Batanopirde
   k. MDL-73147EF;
   l. Metoclopramide
   m. N-3389 (endo-3,9-dimethyl-3,9-diazabicyclo[3,3,1]non-7-yl-1H-indazole-3-carboxamide dihydrochloride),
   n. Y-25130 hydrochloride
   o. MDL 72222
   p. Tropanyl-3,5-dimethylbenzoate
   q. 3-(4-Allylpiperazin-1-yl)-2-quinoxalinecarbonitrile maleate
   r. Zacopride hydrochloride
   s. Mirtazepine (Antidepressant)
6. NK1-receptor antagonists (Neurokinin 1 substance P receptor antagonists)
   a. Aprepitant
   b. MPC-4505
   c. GW597599
   d. MPC-4505
   e. GR205171 (a selective tachykinin NK1 receptor antagonist)
   f. L-759274
   g. SR 140333
   h. CP-96,345
   i. BIIF 1149, NKP 608C, NKP 608A, CGP 60829, SR 140333 (Nolpitantium besilate/chloride), LY 303870 (Lanepitant), MDL-105172A, MDL-103896, MEN-11149, MEN-11467, DNK333A, YM-49244, YM-44778, ZM-274773, MEN-10930, S-19752, Neuronorm, YM-35375, DA-5018, MK-869, L-754030, CJ-11974, L-758298, DNK-33A, 6b-I, CJ-11974.
   j. Benserazide and carbidopa
   k. TAK-637 [(aR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthyridine-6,13-dione]
   l. PD 154075 ([(2-benzofuran)-CH2OCO]-(R)-alpha-MeTrp-(S)—NHCH(CH3)Ph)
   m. FK888, chemical modification of the parent compound, (D-Pro4, DTrp7, 9, 10, Phe11) SP4-11.
7. Antidopaminergic agents/dopamine receptor antagonists
   a. Domperidone
   b. Prochlorperazine
   c. Metoclopramide
   d. Chlorpromazine (Thorazine)
   e. Droperidol (Inapsine)
   f. Promethazine (Phenergan)
8. Benzodiazapines (Valium® and others)
9. Non-psychoactive cannabinoids,
   a. Cannabidiol (CBD)
   b. Cannabidiol dimethylheptyl (CBD-DMH)
   c. Tetra-hydro-cannabinol (THC)
   d. Cannabinoid agonists such as WIN 55-212 (a CB1 and CB2 receptor agonist)
   e. Dronabinol (Marinol®)
10. Further cannabinoids
   a. Nabilone (Cesamet)
11. c-9280 (Merck)

A 5HT3-receptor antagonist is particularly preferred. The anti-emetic is used in an effective amount which is sufficient to either remove or reduce the nausea and/or emesis to an acceptable level.

### A PREFERRED EMBODIMENT

The pharmaceutical composition may also be a kit-in-part further including anti-depressants, such as selective serotonin reuptake inhibitors (SSRIs), serotonin noradrenalin reuptake inhibitors (SNRIs), norepinephrine serotonin reuptake inhibitors (NSRIs), selective noradrenalin reuptake inhibitors, tetracyclic antidepressants, non-selective monoamine reuptake inhibitors including tricyclic antidepressants (TCAs), selective reversible monoamine reuptake inhibitors and antidepressants with other mechanisms of action, e.g. mirtazapin. Examples of SSRIs are citalopram, escitalopram, fluoxetine, fluvoxamine, paroxetine and sertraline. An example of an SNRI is venlafaxine. The kit-in-part may be used for simultaneous, sequential or separate administration. An example of an NSRI is milnacipran.

In a preferred embodiment the pharmaceutical composition may be a kit-in-part.

### Pharmaceutically Acceptable Salts

Pharmaceutically acceptable salts of the present compounds, where they can be prepared, are also intended to be covered by this invention. These salts will be ones which are acceptable in their application to a pharmaceutical use. By that it is meant that the salt will retain the biological activity of the parent compound and the salt will not have untoward or deleterious effects in its application and use in treating diseases.

Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The compounds of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

The pharmaceutical composition of the present invention can include pharmaceutically acceptable salts of the compounds therein. Pharmaceutically acceptable salts include the acid addition salts (formed with the free amino groups of the polypeptide).

Such salts include pharmaceutically acceptable acid addition salts, pharmaceutically acceptable metal salts, ammonium salts and alkylated ammonium salts. Acid addition salts include salts of inorganic acids as well as organic acids. Representative examples of suitable inorganic acids include hydrochloric, hydrobromic, hydriodic, phosphoric, sulfuric and nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, ethylenediaminetetraacetic (EDTA), p-aminobenzoic, glutamic, benzenesulfonic and p-toluenesulfonic acids and the like. Further examples of pharmaceutically acceptable inorganic or organic acid addition salts include the pharmaceutical acceptable salts listed in J. Pharm. Sci. 1977, 66, 2, which is incorporated herein by reference. Examples of metal salts include lithium, sodium, potassium and magnesium salts and the like.

According to the invention organic acid salts of organic acids such as for example acetic acid is preferred.

Examples of ammonium and alkylated ammonium salts include ammonium, methylammonium, dimethylammonium, trimethylammonium, ethylammonium, hydroxyethylammonium, diethylammonium, butylammonium and tetramethylammonium salts and the like.

Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Also included within the scope of compounds or pharmaceutical acceptable acid addition salts thereof in the context of the present invention are any hydrates (hydrated forms) thereof.

The preparation of a pharmacological composition that contains active ingredients dissolved or dispersed therein is well understood in the art. Typically such compositions are prepared as sterile injectables either as liquid solutions or suspensions, aqueous or non-aqueous, however, solid forms suitable for solution, or suspensions, in liquid prior to use can also be prepared. The preparation can also be emulsified.

### Pharmaceutically Acceptable Carriers and Diluents

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

Liquid compositions can also contain liquid phases in addition to and to the exclusion of water. Exemplary of such additional liquid phases are glycerin, vegetable oils such as cottonseed oil, organic esters such as ethyl oleate, and water-oil emulsions.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene or water.

The pharmaceutical compositions formed by combining the compounds of the invention and the pharmaceutical acceptable carriers are then readily administered in a variety of dosage forms suitable for the disclosed routes of administration. The compositions may conveniently be presented in unit dosage form or in multiple dosage form by methods known in the art of pharmacy.

In a still further aspect, the invention relates to a pharmaceutical composition comprising, as an active ingredient, a compound as defined above or a pharmaceutical acceptable salt thereof together with a pharmaceutical acceptable carrier.

### Stabilizers

The active compound of the invention may be unstable, thus the composition preferably contain stabilizers, preservatives or conservatives to increase the stability of the compounds.

A pH-buffering agent may be used to stabilize the active compound of the composition. The buffering agent may be acetate, carbonate, bicarbonate, phosphate, citrate, tris or hepes. In a preferred embodiment the buffering agent is acetate.

According to the invention the composition preferably has a pH between 2.0 and 9.0, or such as between 2.5 and 8.0, or such as 3.0 and 7.0, or such as between 3.5 and 6.0, or such as between 3.5 and 5.0 or such as between 4.0 and 5.5, or such as between 4.0 and 5.0, or such as between 4.0 and 4.5. Preferably the pH of the compositions is less than 6, preferably less than 5.5, preferably less than 5, preferably less than 4.8, preferably less than 4.6, preferably less than 4.4, preferably less than 4.2.

Tween 20, Tween 60, Tween 80, Span 80, cetostearyl alcohol, benzyl alcohol, myristyl alcohol, glyceryl mono-stearate, manitol, polysorbates and sodium lauryl sulphate are possible stabilizers.

In a preferred embodiment manitol may be used as stabilizers

Tween 60, Span 80, cetostearyl alcohol, benzyl alcohol, myristyl alcohol, glyceryl mono-stearate, mannitol and sodium lauryl sulphate are possible stabilizers.

In a preferred embodiment mannitol may be used as stabilizer.

For the preparation of a lyophilised composition ad lyoprotectant may be used to stabilize the active ingredient (Townsend and DeLuca, “Use of lyoprotectants in the freeze-drying of a model protein, ribonuclease A” Journal of Parenteral Science & Technology 42 (6): 190-199 (November-December 1988)).

The lyoprotectant may preferably be a sugar such as sucrose or trehalose such as sucrose, dextran, or hydroxypropyl-/142-cyclodextrin.

### Transport Molecules

Transport molecules act by having incorporated into or anchored to it the compound according to the invention. Any suitable transport molecules known to the skilled person may be used. Examples of transport molecules may be liposomes, micelles, and/or microspheres.

A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028, all of which are incorporated herein by reference.

Micelles are formed by surfactants (molecules that contain a hydrophobic portion and one or more ionic or otherwise strongly hydrophilic groups) in aqueous solution. As the concentration of a solid surfactant increases, its monolayers adsorbed at the air/water or glass/water interfaces become so tightly packed that further occupancy requires excessive compression of the surfactant molecules already in the two monolayers. Further increments in the amount of dissolved surfactant beyond that concentration cause amounts equivalent to the new molecules to aggregate into micelles. This process begins at a characteristic concentration called “critical micelle concentration”.

The shape of micelles formed in dilute surfactant solutions is approximately spherical. The polar head groups of the surfactant molecules are arranged in an outer spherical shell whereas their hydrocarbon chains are oriented toward the centre, forming a spherical core for the micelle. The hydrocarbon chains are randomly coiled and entangled and the micellar interior has a nonpolar, liquid-like character. In the micelles of polyoxyethylated nonionic detergents, the polyoxyethylene moieties are oriented outward and permeated by water. This arrangement is energetically favourable since the hydrophilic head groups are in contact with water and the hydrocarbon moieties are removed from the aqueous medium and partly shielded from contact with water by the polar head groups. The hydrocarbon tails of the surfactant molecules, located in the interior of the micelle, interact with one another by weak van der Waals forces.

The size of a micelle or its aggregation number is governed largely by geometric factors. The radius of the hydrocarbon core cannot exceed the length of the extended hydrocarbon chain of the surfactant molecule. Therefore, increasing the chain length or ascending homologous series increases the aggregation number of spherical micelles. If the surfactant concentration is increased beyond a few percent and if electrolytes are added (in the case of ionic surfactants) or the temperature is raised (in the case of nonionic surfactants), the micelles increase in size. Under these conditions, the micelles are too large to remain spherical and become ellipsoidal, cylindrical or finally lamellar in shape.

Common surfactants well known to one of skill in the art can be used in the micelles of the present invention. Suitable surfactants include sodium laureate, sodium oleate, sodium lauryl sulfate, octaoxyethylene glycol monododecyl ether, octoxynol 9 and PLURONIC F-127 (Wyandotte Chemicals Corp.). Preferred surfactants are non-ionic polyoxyethylene and polyoxypropylene detergents compatible with IV injection such as, TWEEN-80, PLURONIC F-68, n-octyl-beta-D-glucopyranoside, and the like. In addition, phospholipids, such as those described for use in the production of liposomes, may also be used for micelle formation.

### Compositions for Oral Administration

The compounds of the present invention may be formulated in a wide variety of oral administration dosage forms. The pharmaceutical compositions and dosage forms may comprise the compounds of the invention or its pharmaceutically acceptable salt or a crystal form thereof as the active component. The pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, wetting agents, tablet disintegrating agents, or an encapsulating material.

Preferably, the 0.00002% to 2% by weight of a compound or compounds of the invention, with the remainder consisting of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like.

In powders, the carrier is a finely divided solid which is a mixture with the finely divided active component. In tablets, the active component is mixed with the carrier having the necessary binding capacity in suitable proportions and compacted in the shape and size desired. The powders and tablets preferably containing from one to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the composition of the active compound with encapsulating material as carrier providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container aseptically. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Also included are solid form preparations which are intended to be converted, shortly before use, to liquid form preparations for oral administration. Such liquid forms include solutions, suspensions, and emulsions. These preparations may contain, in addition to the active component, colorants, flavours, stabilizers, buffers, artificial and natural sweeteners, dispersants, thickeners, solubilizing agents, and the like.

Other forms suitable for oral administration include liquid form preparations including emulsions, syrups, elixirs, aqueous solutions, aqueous suspensions, toothpaste, gel dentrifrice, chewing gum, or solid form preparations which are intended to be converted shortly before use to liquid form preparations. Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavours, stabilizing and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavours, stabilizers, buffers, artificial and natural sweeteners, dispersants, thickeners, solubilizing agents, and the like.

### Compositions for Parenteral Administration

The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water. Aqueous solutions should be suitably buffered if necessary, and the liquid diluents first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

In a preferred embodiment of the invention, the composition comprising PYY or a functional equivalent thereof or a salt thereof, is a lyophilised composition and the composition may further comprise a solvent. In another embodiment the composition is a solution of PYY or a functional equivalent thereof according to the invention or a salt thereof. Preferably, the solvent may be any suitable solvents, such as described herein, and preferably the solvent is saline or a physiological buffer like phosphate buffer.

The pharmaceutical composition comprises PYY or a functional equivalent thereof or a pharmaceutically acceptable salt thereof, (and for example antigenic epitopes and protease inhibitors). Such compositions can be prepared in water or saline, and optionally mixed with a nontoxic surfactant. Compositions for intravenous or intraarterial administration may include sterile aqueous solutions that may also contain buffers, liposomes, diluents and other suitable additives.

Oils useful in parenteral compositions include petroleum, animal, vegetable, or synthetic oils. Specific examples of oils useful in such compositions include peanut, soybean, sesame, cottonseed, corn, olive, petrolatum, and mineral. Suitable fatty acids for use in parenteral compositions include oleic acid, stearic acid, and isostearic acid. Ethyl oleate and isopropyl myristate are examples of suitable fatty acid esters.

Suitable soaps for use in parenteral compositions include fatty alkali metal, ammonium, and triethanolamine salts, and suitable detergents include (a) cationic detergents such as, for example, dimethyl dialkyl ammonium halides, and alkyl pyridinium halides; (b) anionic detergents such as, for example, alkyl, aryl, and olefin sulfonates, alkyl, olefin, ether, and monoglyceride sulfates, and sulfosuccinates, (c) non-ionic detergents such as, for example, fatty amine oxides, fatty acid alkanolamides, and polyoxyethylenepolypropylene copolymers, (d) amphoteric detergents such as, for example, alkyl-beta-aminopropionates, and 2-alkyl-imidazoline quaternary ammonium salts, and (e) mixtures thereof.

The parenteral compositions typically will contain from about 0.5 to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such compositions will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral compositions can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The pharmaceutical dosage forms suitable for injection can include sterile aqueous solutions or dispersions comprising the active ingredient that are adapted for administration by encapsulation in liposomes. In all cases, the ultimate dosage form must be sterile, fluid and stable under the conditions of manufacture and storage.

Sterile injectable solutions are prepared by incorporating PYY or a functional equivalent thereof or pharmaceutically acceptable salt thereof in the required amount in the appropriate solvent with several of the other ingredients enumerated above, as required, followed by filter sterilization.

### Compositions for Topical Administration

The compounds of the invention can also be delivered topically. Regions for topical administration include the skin surface and also mucous membrane tissues of the rectum, nose, mouth, and throat. Compositions for topical administration via the skin and mucous membranes should not give rise to signs of irritation, such as swelling or redness.

The topical composition may include a pharmaceutically acceptable carrier adapted for topical administration. Thus, the composition may take the form of a suspension, solution, ointment, lotion, cream, foam, aerosol, spray, suppository, implant, inhalant, tablet, capsule, dry powder, syrup, balm or lozenge, for example. Methods for preparing such compositions are well known in the pharmaceutical industry.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or colouring agents. Compositions suitable for topical administration in the mouth include lozenges comprising active agents in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Creams, ointments or pastes according to the present invention are semi-solid compositions of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The composition may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

The pharmaceutical agent-chemical modifier complexes described herein can be administered transdermally. Transdermal administration typically involves the delivery of a pharmaceutical agent for percutaneous passage of the drug into the systemic circulation of the patient. The skin sites include anatomic regions for transdermally administering the drug and include the forearm, abdomen, chest, back, buttock, mastoidal area, and the like.

Transdermal delivery is accomplished by exposing a source of the complex to a patient's skin for an extended period of time. Transdermal patches have the added advantage of providing controlled delivery of a pharmaceutical agent-chemical modifier complex to the body. See Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987). Such dosage forms can be made by dissolving, dispersing, or otherwise incorporating the pharmaceutical agent-chemical modifier complex in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

A variety of types of transdermal patches will find use in the methods described herein. For example, a simple adhesive patch can be prepared from a backing material and an acrylate adhesive. The pharmaceutical agent-chemical modifier complex and any enhancer are formulated into the adhesive casting solution and allowed to mix thoroughly. The solution is cast directly onto the backing material and the casting solvent is evaporated in an oven, leaving an adhesive film. The release liner can be attached to complete the system.

Alternatively, a polyurethane matrix patch can be employed to deliver the pharmaceutical agent-chemical modifier complex. The layers of this patch comprise a backing, a polyurethane drug/enhancer matrix, a membrane, an adhesive, and a release liner. The polyurethane matrix is prepared using a room temperature curing polyurethane prepolymer. Addition of water, alcohol, and complex to the prepolymer results in the formation of a tacky firm elastomer that can be directly cast only the backing material.

A further embodiment of this invention will utilize a hydrogel matrix patch. Typically, the hydrogel matrix will comprise alcohol, water, drug, and several hydrophilic polymers. This hydrogel matrix can be incorporated into a transdermal patch between the backing and the adhesive layer.

The liquid reservoir patch will also find use in the methods described herein. This patch comprises an impermeable or semipermeable, heat sealable backing material, a heat sealable membrane, an acrylate based pressure sensitive skin adhesive, and a siliconized release liner. The backing is heat sealed to the membrane to form a reservoir which can then be filled with a solution of the complex, enhancers, gelling agent, and other excipients.

Foam matrix patches are similar in design and components to the liquid reservoir system, except that the gelled pharmaceutical agent-chemical modifier solution is constrained in a thin foam layer, typically a polyurethane. This foam layer is situated between the backing and the membrane which have been heat sealed at the periphery of the patch.

For passive delivery systems, the rate of release is typically controlled by a membrane placed between the reservoir and the skin, by diffusion from a monolithic device, or by the skin itself serving as a rate-controlling barrier in the delivery system. See U.S. Pat. Nos. 4,816,258; 4,927,408; 4,904,475; 4,588,580, 4,788,062; and the like. The rate of drug delivery will be dependent, in part, upon the nature of the membrane. For example, the rate of drug delivery across membranes within the body is generally higher than across dermal barriers. The rate at which the complex is delivered from the device to the membrane is most advantageously controlled by the use of rate-limiting membranes which are placed between the reservoir and the skin. Assuming that the skin is sufficiently permeable to the complex (i.e., absorption through the skin is greater than the rate of passage through the membrane), the membrane will serve to control the dosage rate experienced by the patient.

Suitable permeable membrane materials may be selected based on the desired degree of permeability, the nature of the complex, and the mechanical considerations related to constructing the device. Exemplary permeable membrane materials include a wide variety of natural and synthetic polymers, such as polydimethylsiloxanes (silicone rubbers), ethylenevinylacetate copolymer (EVA), polyurethanes, polyurethane-polyether copolymers, polyethylenes, polyamides, polyvinylchlorides (PVC), polypropylenes, polycarbonates, polytetrafluoroethylenes (PTFE), cellulosic materials, e.g., cellulose triacetate and cellulose nitrate/acetate, and hydrogels, e.g., 2-hydroxyethylmethacrylate (HEMA).

Other items may be contained in the device, such as other conventional components of therapeutic products, depending upon the desired device characteristics. For example, the compositions according to this invention may also include one or more preservatives or bacteriostatic agents, e.g., methyl hydroxybenzoate, propyl hydroxybenzoate, chlorocresol, benzalkonium chlorides, and the like. These pharmaceutical compositions also can contain other active ingredients such as antimicrobial agents, particularly antibiotics, anesthetics, analgesics, and antipruritic agents.

### Compositions for Administration as Suppositories

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The active compound may be formulated into a suppository comprising, for example, about 0.5% to about 50% of a compound of the invention, disposed in a polyethylene glycol (PEG) carrier (e.g., PEG 1000 [96%] and PEG 4000 [4%]).

### Compounds for Nasal Administration

The compounds of the present invention may be formulated for nasal administration. The solutions or suspensions are applied directly to the nasal cavity by conventional means, for example with a dropper, pipette or spray. The compositions may be provided in a single or multidose form. In the latter case of a dropper or pipette this may be achieved by the patient administering an appropriate, predetermined volume of the solution or suspension. In the case of a spray this may be achieved for example by means of a metering atomizing spray pump.

### Compounds for Aerosol Administration

The compounds of the present invention may be formulated for aerosol administration, particularly to the respiratory tract and including intranasal administration. The compound will generally have a small particle size for example of the order of 5 microns or less. Such a particle size may be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

Compositions administered by aerosols may be prepared, for example, as solutions in saline, employing benzyl alcohol or other suitable preservatives, absorption promoters to enhance bioavailability, employing fluorocarbons, and/or employing other solubilizing or dispersing agents.

### Combinations

It is further envisaged that the composition of the present invention can be administered in combination with a second active ingredient. Said second active ingredient may be co-formulated with the other compound(s) according to the invention.

It is further envisaged that the active ingredient of the present invention can be administered in combination with a second active ingredient. By “in combination” is meant that said composition may be co-formulated with other compounds in the same composition, and/or that said second active ingredient and/or pharmaceutical composition(s) are administered before, during (including concurrently with) and/or after administration of the compositions of the present invention.

Thus an embodiment of the present invention relates to a composition comprising PYY or a functional equivalent there of and a second active ingredient.

Thus an embodiment of the present invention relates to a composition comprising PYY or a functional equivalent thereof as defined herein and a second active ingredient. Said second active ingredient may be selected from the group of; anti-hypertensives including (but not limited to) beta-adrenoceptor antagonists, alpha-adrenoceptor antagonists, calcium antagonists, angiotensin converting enzyme (ACE) inhibitors, angiotensin II receptor antagonists and diuretics (including, but not limited to, thiazide diuretics and loop diuretics), insulin, DPP-IV inhibitors, anti-depressant medications including (but not limited to) selective serotonin reuptake inhibitors (SSRIs), mianserine and mirtazapine, classical as well as atypical antipsychotic drugs (neuroleptics), corticosteroids as well as any other drug(s) that may increase body weight or body fat mass or that may be indicated for the treatment of hypertension, overweight, obesity, the metabolic syndrome (syndrome X) and/or diabetes mellitus.

### Administration

Furthermore are provided in the scope of the present invention methods of treatment of an individual in need thereof, comprising administering to said individual an effective amount of one or more of the pharmaceutical compositions described herein. Said individual is preferably suffering, or at risk of, one or more of the health problems described earlier herein such as gastrointestinal disorders. By “treatment” is also meant prophylaxis and aftercare, and/or lessening of disease symptoms and/or possible disease prevention and/or cure. Said method of treatment may comprise improving the sense of well-being and the quality of life in an individual. Said method may involve one or more of the combination treatments as disclosed herein.

The pharmaceutical compositions of the invention may be used both prophylactically as well as for therapeutic administration. Thus, the pharmaceutical composition comprising PYY or a functional equivalent there of can be administered to patients in order to prevent the development of a gastrointestinal disorder, in order to minimise the severity of a disorder or to patients already suffering from a disorder. Furthermore the therapeutic method may prevent reoccurrence of disorders of the gastrointestine.

The pharmaceutical composition may be prepared so it is suitable for one or more particular administration methods. The composition according to the invention may be administered parenterally, orally, nasally (inhalation or intranasal application), topically (to the skin or to the eye), rectally using suppositories or by intravaginal absorption.

The pharmaceutical composition comprising PYY or a functional equivalent thereof may be administered to an individual in need thereof.

The composition comprising PYY or a functional equivalent thereof can, according to the invention, be administered parenterally, e.g. by injection. Thus, PYY or a functional equivalent thereof may, according to the invention, be administered parenterally, such as intravenously, intra-arterially, intraperitoneally, intramuscularly, subcutaneously, intranasally or transdermally. The composition may further be administered by inhalation, topical application to the eye, intranasal application, intravaginal absorption, application to the skin, and rectal suppositories. Other drug-administration methods, which are effective to deliver the drug to a target site or to introduce the drug into the bloodstream, are also contemplated.

The pharmaceutical compositions containing PYY or a functional equivalent thereof may be administered intravenously, for example by injection of a unit dose. The term “unit dose” when used in reference to a pharmaceutical composition of the present invention refers to physically discrete units suitable as unitary dosage for the subject, each unit containing a predetermined quantity of active material calculated to produce the desired therapeutic effect in association with the required diluent; i.e. carrier, or vehicle. Alternatively, the pharmaceutical composition may be prepared in a multiple dose form and, by use of the multi-dose delivery device, single doses can be administered when needed. As an alternative to standard injections the pharmaceutical composition may be administrated by infusions, such infusions are preferably short.

The effect of PYY or functional equivalents according to the invention is believed to be mediated via actions of PYY outside the central nervous system, with the exception of the NPY neurons located in the hypothalamus. Thereby PYY or functional equivalents do not affect NPY neurons located elsewhere in the central nervous system.

Accordingly, PYY or functional equivalents capable of binding to the NPY Y2 receptor may circulate in the bloodstream to the receptors in the hypothalamus; however, these molecules should preferably not be capable of crossing the blood-brain barrier, and thereby not be able to enter into other parts of the central nervous system.

The composition according to the invention may be administrated in combination with a second pharmaceutical composition. By “in combination” is meant that said composition may be co-formulated with other compounds in the same composition, and/or that said other compound(s) and/or pharmaceutical composition(s) are administered before, during (including concurrently with) and/or after administration of the compositions of the present invention.

Thus in an embodiment the composition of the invention may be administered in combination with a second pharmaceutical composition. The compositions may be administered simultaneously, either as separate compositions or combined in a unit dosage form, or administered sequentially as two separate pharmaceutical compositions.

In an embodiment the pharmaceutical composition according to the invention is administered in combination with a second pharmaceutical composition selected from the group of pharmaceutical composition comprising; anti-depressants, such as selective serotonin reuptake inhibitors (SSRIs), serotonin noradrenalin reuptake inhibitors (SNRIs), norepinephrine serotonin reuptake inhibitors (NSRIs), selective noradrenalin reuptake inhibitors, tetracyclic antidepressants, non-selective monoamine reuptake inhibitors including tricyclic antidepressants (TCAs), selective reversible monoamine reuptake inhibitors and antidepressants with other mechanisms of action, e.g. mirtazapin. Examples of SSRIs are citalopram, escitalopram, fluoxetine, fluvoxamine, paroxetine and sertraline. An example of an SNRI is venlafaxine. An example of an NSRI is milnacipran.

### Method of Treatment

The method of treatment may aim at reducing the symptoms of the gastrointestinal disorder.

The compositions are preferably administered after sensing of symptoms, such as 5 minutes after sensing of symptoms, such as 10 minutes after sensing of symptoms, such as 15 minutes after sensing of symptoms, such as up to 20 minutes after sensing of symptoms, such as up to 30 minutes after sensing of symptoms, such as up to 45 minutes after sensing of symptoms, such as up to 60 minutes after sensing of symptoms, such as up to 75 minutes after sensing of symptoms, such as up to 90 minutes after sensing of symptoms, such as up to 120 minutes after sensing of symptoms, such as up to 150 minutes after sensing of symptoms, such as up to 180 minutes after sensing of symptoms.

The composition may administered once a day, or every second day or twice a week or once a week. Administration of said pharmaceutical composition may be once a day, or twice a day, or three times a day.

The pharmaceutical composition may be administered prior to a meal, such as within 5 minutes of a meal, such as within 20 minutes of a meal, such as within 60 minutes of a meal.

The pharmaceutical preparations described herein may also be arranged in unit dosage forms. In such a form, the preparation is subdivided into unit doses containing appropriate quantities of the active component. The unit dosage form can be a packaged preparation, the package containing discrete quantities of preparation, such as powders in compartments. In this embodiment the powders may be mixed with a solvent prior to or during use.

In one aspect of the present invention, a suitable dose of the compositions described herein is administered in pharmaceutically effective amounts to an individual in need of such treatment. Herein, “pharmaceutically effective amounts”, is defined as an administration involving a total amount of each active component of the pharmaceutical composition or pharmaceutical composition or method that is sufficient to show a meaningful patient benefit. The term “unit dosage form” as used herein refers to physically discrete units suitable as unitary dosages for human and animal subjects, each unit containing a predetermined quantity of a compound, alone or in combination with other agents, calculated in an amount sufficient to produce the desired effect in association with a pharmaceutically acceptable diluent, carrier, or vehicle. The specifications for the unit dosage forms of the present invention depend on the particular compound or compounds employed and the effect to be achieved, as well as the pharmacodynamics associated with each compound in the host. The dose administered should be an “effective amount” or an amount necessary to achieve an “effective level” in the individual patient.

The pharmaceutical compositions may be administrated by intravenous infusion. The duration of an infusion may be less than 120 minutes, such as less than 100 minutes, such as less than 80 minutes, such as less than 60 minutes, such as less than 40 minutes, such as less than 20 minutes, such as less than 10 minutes or such as less than 5 minutes.

The dosage requirements will vary with the particular drug composition employed, the route of administration and the particular subject being treated. Ideally, a patient to be treated by the present method will receive a pharmaceutically effective amount of the compound not exceeding the maximum tolerated dose (MTD), which is generally no higher than that required before drug resistance develops.

Suitable dosing regimens are preferably determined taking into account factors well known in the art including type of subject being dosed; age, weight, sex and medical condition of the subject; the route of administration; the renal and hepatic function of the subject; the desired effect; and the particular compound employed.

Optimal precision in achieving concentrations of drug within the range that yields efficacy without toxicity requires a regimen based on the kinetics of the drug's availability to target sites. This involves a consideration of the distribution, equilibrium, and elimination of a drug. For the present invention the dosage will vary depending on the compound employed and the mode of administration.

The dosage may be calculated based on the body weight of the subject but in certain situations the dosages may be calculated base on the fat free mass (FFM) of the subject. Thus the dosage may be in concentration equivalent to PYY1-36 or PYY3-36.

For the present invention the dosage will vary depending on the compound employed and the mode of administration. Dosage levels may vary between about 4 ng/kg body weight to 20 μg/kg body weight daily, preferably between about 10 ng/kg body weight to 1 μg/kg body weight, more preferably between 50 to 750 ng/kg body weight. Alternative dosages in relation to FFM may vary between about 5 ng/kg FFM to 25 μg/kg FFM daily, preferably between about 12.5 ng/kg FFM to 1.25 μg/kg FFM, more preferably between 62.5 to 875 ng/kg FFM. To obtain dosages in relation to FFM the dosage/kg bodyweight should be multiplied by the factor 1.25.

The dosage may be administered when needed, such as up to ten times daily, such as one to five times daily, such as two or three times daily, or preferably such as once a day, thus the daily dosage maybe up to 2-5 times the dosages mentioned above. Alternatively, the dosage may be administered less frequently than once daily as described herein above.

A preferred dosage of a composition employed according to the invention is in a concentration equivalent to PYY or a functional equivalent there of from 4 ng to about 20 μg per kg bodyweight, or such as from 10 ng to 1 μg per kg bodyweight, more preferably from 50 to 750 ng per kg bodyweight. The dosage of PYY is preferably 20-200 ng/kg, 20-160 ng/kg, 40-160 ng/kg, 40-120 ng/kg, 40-80 ng/kg, 60-120 ng/kg or such as approximately 60 ng/kg or 80 ng/kg.

The preferred dosages may be in a concentration equivalent to PYY or a functional equivalent there of from 1 pmol/kg to 5 nmol/kg, such as from 5 pmol/kg to 1 nmol/kg, or such as from 20 pmol/kg to 500 pmol/kg alternatively such as from 40 to 160 pmol/kg or such as from 75 to 120 pmol/kg. PYY1-36 is preferably administered in dosages of, such as from 50-400 pmol/kg, such as 80-320 pmol/kg, such as 150-250 pmol/kg, such as about 200 pmol/kg. PYY3-36 is preferably administered in dosages of such as from 30-350 pmol/kg, such as 50-280 pmol/kg, such as 80-200 pmol/kg, such as about 120 pmol/kg. Dosages in relation to FFM may be calculated by multiplying the indicated dosages with 1.25.

In a second embodiment the dosage may be 5-50 pmol/kg, 5-40 pmol/kg, 5 to 30 pmol/kg, 10-40 pmol/kg, 10-30 pmol/kg such as 5 to 25 pmol/kg, such as 5 to 20 pmol/kg, and most preferably 10-20 pmol/kg, 15-30 pmol/kg or approximately 15 pmol/kg or 20 pmol/kg.

The dosages are preferably administrated once a day, or such as two times a day, or such as three times a day, or such as four times a day, or such as five times a day, or such as more than five times a day.

In one preferred embodiment of the present invention, the compositions are administered in dosages of PYY or a functional equivalent from about 400 ng to about 2 mg, more preferably from about 10 μg to about 200 μg, or from about 5 μg to about 250 μg, more preferably from about 20 μg to about 200 μg, more preferably from about 20 μg to about 100 μg. Most preferably the dosage may be 1-20 μg, 2-16 μg, 4-16 μg, 4-12 μg, 4-8 μg, 6-12 μg, 6-10 μg or approximately 8 μg.

In a preferred embodiment the composition is administered in dosages of PYY or a functional equivalent from 100 pmol to 500 nmole, or such from 500 pmol to 100 nmol, or such as from 1 nmol to 50 nmol, or such as from 2 to 25 nmol, or such as from 4 to 20 nmol. Alternatively the preferred dosage may be 0.25-5 nmol, 0.5-4 nmol, 1-4 nmol, 1-3 nmol, 1-2 nmol, 1, 5-3 nmol or more preferably 1.5-2.5 nmol or most preferably approximately 2 nmol. In a further preferred embodiment a PYY1-36 dosages includes such as from 5-40 nmol, such as 8-32 nmol, such as 15-25 nmol, such as about 20 nmol, whereas a dosage of PYY3-36 includes such as from 3-35 nmol, such as 5-28 nmol, such as 8-200 nmol, such as about 12 nmol.

In another embodiment, the PYY or functional equivalent is administered subcutaneously in a dosage of 5-30 pmol/kg, such as 5-25 pmol/kg, such as 5-20 pmol/kg or such as 10-20 pmol/kg bodyweight, in order to achieve an effective level in the individual treated. The presently preferred dosage is 10-20 pmol/kg bodyweight of PYY1-36 or PYY 3-36. In second preferred embodiment the dosages of PYY1-36 is 150-250 pmol/kg and/or the dosages of PYY3-36 is 80-150 pmol/kg.

The dosages of PYY or the functional equivalent is preferably administered once a day, or such as two times a day, or such as three times a day, or such as four times a day, or such as five times a day.

The pharmaceutical preparations described herein may also be arranged in unit dosage forms. In such form, the preparation is subdivided into unit doses containing appropriate quantities of the active component. The unit dosage form can be a packaged preparation, the package containing discrete quantities of preparation, such as powders in compartments. In this embodiment the powders may be mixed with a solvent prior to or during use.

In a preferred embodiment the PYY composition is administrated in unit dosage form, from about 400 ng to about 2 mg of PYY or a functional equivalent thereof, more preferably from about 10 μg to about 200 μg, or from about 5 μg to about 250 μg, more preferably from about 20 μg to about 200 μg, more preferably from about 20 μg to about 100 μg. The unit dosage form may comprise from 100 pmol to 500 nmole, or such from 500 pmol to 100 nmol, or such as from 1 nmol to 50 nmol, or such as from 2 to 25, such as from 4 nmol to 20 nmol. of PYY or a functional equivalent thereof. The compositions are preferably administered once a day, or such as two times a day, or such as three times a day, or such as four times a day, or such as five times a day. In a further preferred embodiment a unit dosage of PYY1-36 includes such as from 5-40 nmol, such as 8-32 nmol, such as 15-25 nmol, such as about 20 nmol, whereas a unit dosage of PYY3-36 includes such as from 3-35 nmol, such as 5-28 nmol, such as 8-200 nmol, such as about 12 nmol.

In certain embodiment the pharmaceutical composition may be administered by infusions. The dosages of PYY or a functional equivalent for infusion may be from 0.01 pmol/kg minute to 500 pmol/kg minute such as from 0.05 pmol/kg minute to 100 pmol/kg minute, or such as from 0.1 pmol/kg minute to 50 pmol/kg minute, or such as from 1 pmol/kg min. to 25 pmol/kg minute.

The compositions are preferably administered after sensing of symptoms, such as 5 minutes after sensing of symptoms, such as 10 minutes after sensing of symptoms, such as 15 minutes after sensing of symptoms, such as up to 20 minutes after sensing of symptoms, such as up to 30 minutes after sensing of symptoms, such as up to 45 minutes after sensing of symptoms, such as up to 60 minutes after sensing of symptoms, such as up to 75 minutes after sensing of symptoms, such as up to 90 minutes after sensing of symptoms, such as up to 120 minutes after sensing of symptoms, such as up to 150 minutes after sensing of symptoms, such as up to 180 minutes after sensing of symptoms.

The PYY composition of the present invention may be administered admixed with a pharmaceutically acceptable carrier or diluent.

It is thereby included that the treatment may be administered during the night. Furthermore, since the “effective level” is used as the preferred endpoint for dosing, the actual dose and schedule can vary, depending on individual differences in pharmacokinetics, drug distribution, and metabolism. The “effective level” can be defined, for example, as the blood or tissue level desired in the patient that corresponds to a concentration of one or more compounds according to the invention.

The effective level may refer to an amount of the active ingredient of the composition according to the invention that is able to obtain certain blood or tissue levels of a desired compound in the patient. The effective level may be the amount of the active ingredient of the composition according to the invention that is able to diminish the symptoms in the patient.

The pharmaceutical composition may be administration during hospitalization. In addition it may be beneficial for the patient if the composition can be self-administered.

In one embodiment of the invention, the compositions of the present invention are self-administered. The pharmaceutical composition may be administered by use of an injection device, for example by the use of a system similar to insulin pens.

In one embodiment of the invention the composition is administered by use of a single or a multi-dose injection device.

The pharmaceutical composition may also be a kit-in-part further including anti-depressants, such as selective serotonin reuptake inhibitors (SSRIs), serotonin noradrenalin reuptake inhibitors (SNRIs), norepinephrine serotonin reuptake inhibitors (NSRIs), selective noradrenalin reuptake inhibitors, tetracyclic antidepressants, non-selective monoamine reuptake inhibitors including tricyclic antidepressants (TCAs), selective reversible monoamine reuptake inhibitors and antidepressants with other mechanisms of action, e.g. mirtazapin. Examples of SSRIs are citalopram, escitalopram, fluoxetine, fluvoxamine, paroxetine and sertraline. An example of an SNRI is venlafaxine. The kit-in-part may be used for simultaneous, sequential or separate administration. An example of an NSRI is milnacipran.

In a preferred embodiment the pharmaceutical composition may be a kit-in-part.

### EXAMPLES

The following examples illustrate the invention without limiting it thereto.

### Example 1

### Binding Assay and Functional Assay

Transfections and tissue culture: COS-7 cells can be grown Dulbecco's Modified Eagle'e Medium 1885 supplemented with 10% fetal calf serum, 2 mM glutamine and 0.01 mg/ml gentamicin. The expression plasmids containing the cDNAs encoding the wild type or the mutated receptors can be transiently expressed after transfection according to the calcium phosphate precipitation method and assay can be performed 48 hour after transfection.

Binding assay: One day after transfection the cells will be transferred and seeded in multi-well plates for assay. The number of cells to be plated per well will be chosen so as to obtain 5 to 10% binding of the radioligand added. Two days after transfection the cells will be assayed in competition binding assays using ¹²⁵I-PYY(3-36) as a tracer. Radioligand will be bound in a buffer composed of 0.5 ml of 50 mM Hepes buffer, pH 7.4, supplemented with 1 mM CaCl₂, 5 mM MgCl₂, and 0.1% BSA, and displaced in a dose dependent manner by unlabelled ligands. The assay will be performed in duplicate for 3 hours at 4° C., and stopped by washing twice in the buffer. Cell associated, receptor bound radioligand will be determined by the addition of lysis buffer (48% urea, 2% NP-40 in 3M acetic acid). The concentration of radioligand in the assay corresponds to a final concentration of approximately 20 pM.

### Functional Assay.

COS-7 can be cultured as described above and contransfections can be performed. The activation of Phospholipase C by chimeric G-proteins (Conklin B) formed between both Gαq and Gαi the Y2 receptor can be measured through the inositol phosphate (IP) turnover in the cell. The IP turnover may be recorded by use of following assay:

One day after transfection COS-7 cells are incubated for 24 hours with 5:Ci of [³H]-myo-inositol (Amersham, PT6-271) in 1 ml medium supplemented with 10% fetal calf serum, 2 mM glutamine and 0.01 mg/ml gentamicin per well. Cells are washed twice in buffer, 20 mM HEPES, pH 7.4, supplemented with 140 mM NaCl, 5 mM KCl, 1 mM MgSO₄, 1 mM CaCl₂, 10 mM glucose, 0.05% (w/v) bovine serum; and are incubated in 0.5 ml buffer supplemented with 10 mM LiCl at 37EC for 30 min. The indicated curves are furthermore incubated with adenosine deaminase ADA (200 U/mg, Boeringer Mannheim, Germany) for 30 min in a concentration of 1 U/ml. After stimulation with various concentrations of peptide for 45 min at 37 C, cells will be extracted with 10% ice-cold perchloric acid followed by incubation on ice for 30 min. The resulting supernatants are neutralized with KOH in HEPES buffer, and the generated [³H]-inositol phosphate is purified on Bio-Rad AG 1-X8 anion-exchange resin. Determinations will be made in duplicates.

### Example 2

### Synthetic Production of PYY and Functional Equivalents Thereof

The polypeptide of the present invention may be produced by a conventional peptide synthesis method.

Amino acid derivatives and synthesis reagents, can be obtained from commercial sources. Peptide chain extension is performed by mainly using Applied Biosystem 433A synthesizer produced by Perkin Elmer, and a protected peptide derivative-resin is constructed by the Boc or Fmoc method. The protected peptide resin obtained by the Boc method is deprotected with anhydrous hydrogen fluoride (HF) in the presence of p-cresol thereby releasing the peptide, which is then purified. The protected peptide resin obtained by the Fmoc method is deprotected with trifluoroacetic acid (TFA) or dilute with TFA containing various scavengers, and the released peptide is purified. Purification is performed in reversed phase HPLC on a C4 or C18 column. The purity of the purified product is confirmed by reverse phase HPLC, and its structure is confirmed by amino acid composition analysis and mass spectrometry.

### Example 3

### Measurements of PYY Plasma Levels

The experiment is performed by subcutaneous injections of placebo and 4 escalating doses of PYY1-36 or PYY3-36 as set out in table 1. The dosages of PYY is calculated base on the fat free mass (FFM) of the subject.

TABLE 1
Dosages of PYY and number of subjects (n)

| Stof | Dosis, pmol/kg FFM | Number of subjects (n) |
|---|---|---|
| PYY1-36 | 12.5 | 2 |
| | 25 | 2 |
| | 50 | 12 |
| | 100 | 12 |
| | 150 | 10 |
| | 200 | 10 |
| PYY3-36 | 12.5 | 2 |
| | 25 | 12 |
| | 50 | 12 |
| | 75 | 10 |
| | 100 | 12 |

The results are presented as mean ±SE, paired t-test (SAS) and repeated measures (SAS).

The PYY injections is performed at time 0 minutes and the plasma concentrations of PYY upon PYY1-36 and PYY3-36 administration is measured at t=0, 15, 30, 45, 60, 75, 90, 120, 150, 180, 210 and 240 minutes.

### PYY Assay

The plasma concentration of PYY is measured using radioimmunoassay of PYY. The assays are performed using PYY antiserum (code no. 8412-5) (EuroDiagnostica, Malmoe, Sweden). The antiserum recognizes both human PYY 1-36 and PYY 3-36. Synthetic human PYY 1-36 (Peninsula, Merseyside, UK) and porcine ¹²⁵I-PYY (code no. IM259) is purchased from Amersham Biosciences, Buckinghamshire UK) for use as standards. Detection limit of the assay is below 2 pmol/l and 50% inhibition is obtained with 40 pmol/l PYY. Recovery of PYY added to plasma in concentrations between 5 and 50 pmol/l deviates less than 15% from expected values. Intra-assay coefficient of variation is below 5%. The antiserum shows no cross reaction with human NPY or human PP in concentrations up to 500 pmol/l.

The results are shown in FIGS. 2 and 3.

### Example 4

### In Vivo Measuring of the Effect of PYY on Migrating Myoelectric Complexes

### Method

Rats are supplied with bipolar electrodes at 5 (duodenum), 15 and 25 cm (jejunum) distal to pylorus for electromyography of small intestine. The natural ligands peptide YY1-36 and peptide YY 3-36 are infused IV at doses of 0.5-400 pmol/kg min for 60 min. The mechanisms of action are studied in bilaterally vagotomised animals after pre-treatment with N-nitro-L-arginine (L-NNA) 1 mg/kg and guanethidine 3 mg/kg.

The experimental protocol was approved by the local Ethics committee for animal experimentation in northern Stockholm, Sweden.

### Materials

Male Sprague-Dawley rats (B&K, Sollentuna, Sweden) weighing 300-350 g were used for the study.

### Preparation of Rats for Electromyography.

The rats are anaesthetized with pentobarbital (Apoteksbolaget, Umeå, Sweden) using 50 mg/kg intraperitoneally. Through a midline incision, three bipolar stainless steel electrodes (SS-5T, Clark Electromedical Instruments, Reading, UK) are implanted into the muscular wall of the small intestine 5 (D), 15 (J1), and 25 (J2) cm distal to the pylorus. All animals are supplied with a jugular vein catheters for administration of drugs. The electrodes and catheters are tunneled subcutaneously to exit at the back of the animal's neck. After surgery the animals are housed singly and allowed to recover for at least 7 days before experiments are undertaken. During recovery the rats are trained to accept experimental conditions. Experiments are then carried out in conscious animals after an 8-h fasting period in wire-bottomed cages with free access to water. During the experiments, the rats are placed in Bollman cages. The electrodes are connected to an EEG preamplifier (7P5B) operating a Grass Polygraph 7B (Grass Instruments, Quincy, Mass.). The time constant is set at 0.015 s and the low and high cut-off frequencies is set at 10 and 35 Hz, respectively.

### Design of Electromyography Studies.

All experiments are started with a control recording of basal myoelectric activity with four activity fronts propagated over all three recording sites during a period of 60 min.

Infusions are started immediately after the fourth activity front has passed the first electrode site using a microinjection pump (CMA 100, Carnegie Medicine, Stockholm, Sweden).

In a first series of experiments, the natural ligands PYY1-36 or PYY3-36 at doses of 0.5 to 400 pmol/kg min is administered intravenously for 60 min.

A second series of experiments is performed with administration of a NO synthase inhibitor N^{ω}-nitro-L-arginine (L-NNA) at 1 mg/kg given as a bolus injection 45 min before infusion of PYY1-36 or PYY3-36 at 100 pmol/kg min.

In a third series of experiments 3 mg/kg guanethidine is administered on day one and the following day the effect of PYY1-36 or PYY3-36 at 100 pmol/kg min is studied.

In a forth series of experiments the effect of infusion of PYY1-36 or PYY3-36 at 100 pmol/kg min is studied in vagotomized and sham-vagotomized animals.

### Drugs

RatPYY1-36 and ratPYY3-36 is purchased from Neosystem (Strasbourg, France), L-NNA from Sigma-Aldrich, (St. Louis, Mo., USA) and guanethidine from Apoteksbolaget (Stockholm, Sweden).

### Data Analysis

The main characteristic feature of myoelectric activity of the small intestine in the fasted state, the activity front, or phase III of MMC, is identified as a period of clearly distinguishable intense spiking activity with an amplitude at least twice that of the preceding baseline, propagating aborally through the whole recording segment and followed by a period of quiescence, phase I of MMC.

The percent of the recording periods occupied by phase III activity fronts is calculated for stimulatory periods (60 min). The effects of the peptides is expressed as percent of time occupied by activity fronts.

Dose-response curves are generated by Graph-Pad Prism 4 (GraphPad, San Diego, Calif., USA). Data are expressed as mean ±SEM. Differences between individual data groups are determined using ANOVA, followed by Dunnet's test or students T-test, as appropriate, with statistical significance at p<0.05.

### Example 5

### Clinical Protocol Gastro Intestinal Disorders

45 subjects that meet the proposed diagnostic criteria of IBS or functional dyspepsias are included in the study.

The study is performed in a double-blinded, placebo-controlled fashion. Subjects are divided into three groups (n=15 in each), groups A, B and C. The subjects are given diaries where they note their symptoms (bowel habits/pain/nausea etc) and eating information. This initial phase of the study is 4 weeks (“Run-in Phase”), after which the subjects start treatment with one of three regimens, as defined below. The subjects keep diaries where they note the timing, type and severity of experienced symptoms throughout the treatment phase. The treatment duration is 4 weeks (“Treatment Phase”).

Dosing: The subjects of group A receive subcutaneous placebo injections (NaCl) three times daily (distributed evenly over the hours awake). The subjects of group B receive 60 pmol/kg body weight of PYY 3-36 s.c. three times daily and the subjects of group C receive 100 pmol/kg body weight of PYY 3-36 s.c. three times daily.

The subjects' diaries are reviewed by the investigators and the severity of symptoms is determined in relation to food intake and timing.

The effect on symptoms is evaluated by comparing the results during the 4-week Treatment Phase with the results during the Run-in Phase for each subject, and then statistically comparing the result from group B with group A, and the result from group C with group A.

### Example 6

### Effects of PYY on Pain Sensation and the Spontaneous Motor Activity in the Rectum

12 patients (men or women between 18 and 60 years of age) meeting the Rome II criterias is included in the study.

### Conduct of the Study

All patients will come to the clinic after a 12-hour fasting period. Before the study patients will be given a tap water enema.

A probe (Barostat bag) will be inserted in the rectum and connected to an electronic barostat that is programmed to automatically perform distensions with fixed time lags and bag pressure increments.

The study is performed as 3-way cross-over study with approx. 7 days between treatments.

### A) S.c. administration of PYY1-36 in 0.9% NaCl solution.

### B) S.c. administration of PYY3-36 in 0.9% NaCl solution.

### C) S.c. administration of saline (0.9% NaCl solution).

After administration of PYY or saline the distension according to a fixed protocol will begin. The patients will note the discomfort and pain using a visual analog scale (VAS). Patients will be given control over the protocol by their ability to deflate the bag instantaneously at any time of significant discomfort or pain.

The threshold pressure and volume will be written down.

After a fixed period of time e.g. 20 or 30 minutes a saline meal with fixed energy content will be given p.o.

Hereafter a second distension similar to the first will begin.

In between to two distensions a fixed low pressure will be kept in the barostat bag. This will allow recording of the volume in the bag as a measure of the motor activity in the rectum.

### Primary Endpoint

Comparison of the pain threshold with or without food intake in patients with IBS and the effect of PYY compared to placebo.

### Second Endpoint

Study of the physiological effects of PYY on the spontaneous motor activity in the rectum compared to placebo.

The effect on symptoms is evaluated by statistically comparing the result from group B with group A, and the result from group C with group A.

Statistical analysis is performed by a non-parametric analysis of variance (Kruskal-Wallis test).

1. A method of treating a functional gastrointestinal disorder, said method comprising administering to a subject in need thereof, a therapeutically effective amount of a composition comprising PYY or a functional equivalent thereof or a pharmaceutical acceptable salt thereof, said disorder or said administration being characterized by:
(a) the predominant symptom being pain and/or
(b) the predominant symptom being abdominal pain and/or
(c) the predominant symptom being abdominal discomfort and/or
(d) the predominant bowel habit being constipation and/or
(e) the predominant bowel habit being alternating diarrhoea and constipation and/or
(f) the treatment being administered parenterally.

2. (canceled)

3. The method according to claim 1 for the treatment of abdominal pain

4. The method according to claim 3 for the treatment of visceral pain.

5. The method according to claim 1, wherein the functional gastrointestinal disorder is irritable bowel syndrome (IBS).

6. The method according to claim 1, wherein the functional gastrointestinal disorder is functional dyspepsia (FD).

7. The method according to claim 6 for the relief of the symptom(s)
a. sensation of fullness and/or
b. inability to finish a normal sized meal and/or
c. pain after food intake and/or
d. nausea and/or
e. vomiting and/or
f. bloating and/or
g. belching and/or
h. regurgitation and/or
i. epigastric pain and/or
j. feeling of distention and/or
k. exess flatus
and any combination of the above.

8. The method according to claim 1, wherein the symptoms are predominantly upper GI symptoms.

9. The method according to claim 1, wherein the symptoms are predominantly lower GI symptoms.

10. The method according to claim 1, wherein said composition comprise human PYY

11. The method according to claim 1, wherein said composition comprise human PYY1-36 identified by SEQ ID NO 1.

12. The method according to claim 1, wherein said composition comprise human PYY3-36 identified by SEQ ID NO 2.

13. The method according to claim 1, wherein the pH of the composition is between 2.0 and 9.0

14. The method according to claim 1, wherein the pharmaceutical is formulated for parenteral administration.

15. The method according to claim 1, wherein the pharmaceutical composition is formulated for subcutaneous administration.

16. The method according to claim 1, wherein the pharmaceutical composition is for intranasal administration.

17. The method according to claim 1, wherein the composition comprises a second active ingredient.

18. The method according to claim 17, wherein the second pharmaceutical composition is selected from the group of anti-depressants consisting of SSRIs, SNRIs non-selective monoamine reuptake inhibitors selective reversible monoamine reuptake inhibitors and mirtazapin.

19. The method according to claim 17, wherein the second pharmaceutical composition is an anti-emetic drug.

20. The method according to claim 19 for the treatment of irritable bowel syndrome (IBS) and/or functional dyspepsia (FD).

21. (canceled)

22. The method according to claim 18, wherein the antidepressant is a SSRI selected from the group consisting of: citalopram, escitalopram, fluoxetine, fluvoxamine, paroxetine and sertraline.

23. The method according to claim 18, wherein the antidepressant is a SNRI.

24. The method according to claim 23, wherein the antidepressant is venlafaxine.

25. The method according to claim 18, wherein the antidepressant is a NSRIs.

26. The method according to claim 18, wherein the antidepressant is a tetracyclic antidepressant.

27. The method according to claim 18, wherein the antidepressant is a non-selective monoamine reuptake inhibitor.

28. The method according to claim 27, wherein the antidepressant is a tricyclic antidepressant.

29. The method according to claim 18, wherein the antidepressant is a selective reversible monoamine reuptake inhibitor.

30. The method according to claim 18, wherein the antidepressant is mirtazapin.
